(19) United States
(12) Patent Application Publication
(10) Pub. No.: US 2001/0041870 A1
(43) Pub. Date: Nov. 15, 2001

(54) IMPLANTABLE DEVICE FOR ACCESS TO A TREATMENT SITE

(76) Inventors: EDWARD M. GILLIS, CUPERTINO, CA (US); FELIX THEEUWES, LOS ALTOS HILLS, CA (US)

Correspondence Address: CAROL L. FRANCIS, BOZICEVIC, FIELD & FRANCIS LLP, 200 MIDDLEFIELD ROAD, SUITE 200, MENLO PARK, CA, 94025, US

(21) Appl. No.: 09/282,921

(22) Filed: Mar. 31, 1999

Related U.S. Application Data
(60) Provisional application No. 60/123,474, filed on Mar. 9, 1999.

(51) Int. Cl. A61M 5/178

(52) U.S. Cl. 604/164.09; 604/530

(57) ABSTRACT

The present invention provides an implantable guide for access to a treatment site. The implantable guide comprises a proximal end, a distal end, and a guide body defining a lumen, and can optionally comprise a stable positioning element for stably positioning a drug delivery device within the guide. The guide can be provided in connection with a drug delivery device. In use, the guide is implanted within a subject so as to provide a conduit through which a drug delivery device can be retrievably introduced to facilitate delivery of drug to a treatment site within a subject at a site distal to an accessible implantation site. The drug delivery device is then positioned within the guide lumen to provide for delivery of drug from the drug delivery to the desired treatment site.

### CROSS-REFERENCE TO RELATED APPLICATIONS

This application is a continuation-in-part of U.S. application Ser. No. 60/123,474, filed Mar. 9, 1999, which application is incorporated herein by reference.

### FIELD OF THE INVENTION

This invention relates generally to implantable devices and methods of use relating to same, particularly to site-specific drug delivery.

### BACKGROUND OF THE INVENTION

Few therapeutic regimen involve administration of a single dose of a selected drug. Instead, most therapies require administration of multiple doses. Where the therapy requires parenteral delivery of the drug, the patient can be subjected to the substantial discomfort and inconvenience of repeated injections. This can be particularly problematic where the condition or disease to be treated requires long-term therapy. The repeated injections required for such long-term therapy not only meet with difficulties associated with patient compliance, but also can lead to collapse of veins and substantial tissue damage. Parenteral drug delivery typically also requires administration of a bolus of drug in order to provide for an effective drug concentration at the desired treatment site and/or to provide for an adequate systemic levels for an acceptable period of time (e.g., as in treatment of diabetes with insulin). Delivery of a drug bolus not only requires delivery of a greater amount of drug, thus driving up the cost of therapy, but can also be associated with undesirable side effects.

One approach for avoiding at least some of the problems inherent in long-term drug delivery involves the use of an implantable drug delivery device. Examples of such implantable drug delivery devices include implantable diffusion systems (see, e.g., subdermal implants (such as NORPLANT™) and other such systems, see, e.g., U.S. Pat. Nos. 5,756,115; 5,429,634; 5,843,069). These implants generally operate by simple diffusion, e.g., the active agent diffuses through a polymeric material at a rate that is controlled by the characteristics of the active agent formulation and the polymeric material. An alternative approach involves the use of biodegradable implants, which facilitate drug delivery through degradation of the implant material that contains the drug (see, e.g., U.S. Pat. No. 5,626,862). Alternatively, the implant may be based upon an osmotically-driven device to accomplish controlled drug delivery (see, e.g., U.S. Pat. Nos. 3,987,790, 4,865,845, 5,057,318, 5,059,423, 5,112,614, 5,137,727, 5,234,692; 5,234,693; and 5,728,396). These osmotic pumps generally operate by imbibing fluid from the outside environment and releasing corresponding amounts of the therapeutic agent.

While such drug delivery devices avoid the need for repeated injection often associated with long-term drug therapies, the treatment site to which drug delivery is desired is often not amenable to insertion of such an implant. For example, while such implants may be useful in delivering a chemotherapeutic to a localized breast tumor, there are many sites within the body (e.g., a site deep within a subject's body) or to a site that is particularly fragile or sensitive (e.g., the spinal cord) where the implant cannot be easily or practically inserted. Although these implants could instead be used to deliver the drug systemically, systemic delivery is often not an acceptable form of long-term drug delivery. Many therapeutic drugs are highly toxic and/or may cause dangerous side effects. Moreover, systemic administration normally requires administration of higher doses in order to provide an effective concentration at a desired treatment site, making therapies more likely to be associated with side-effects and more expensive.

Implantable infusion devices having an associated drug delivery catheter avoid at least some of the problems associated with the implantable diffusion systems and biodegradable systems described above. Implantable infusion devices can control delivery of drug by, for example, use of a programmable pump that controls release of the drug from a reservoir at a certain rate to a desired treatment site (see, e.g., U.S. Pat. Nos. 4,692,147; 5,713,847; 5,711,326; 5,458,631; 4,360,019; 4,487,603; and 4,715,852). Alternatively, implantable infusion devices can control drug delivery by means of a rate-limiting membrane positioned between the drug reservoir and the delivery catheter (see, e.g., U.S. Pat. No. 5,836,935), or by only releasing drug from the reservoir upon application of pressure to a subcutaneously positioned device (see, e.g., U.S. Pat. No. 4,816,016; 4,405,305). Implantable infusion devices have been described for intravenous, intra-arterial, intrathecal, intraperitoneal, intraspinal and epidural drug delivery. In general, these pumps are usually surgically inserted into a subcutaneous pocket of tissue (e.g., in the lower abdomen), and a catheter attached to the pump is positioned at a desired treatment site (see, e.g., U.S. Pat. No. 4,692,147).

While implantable infusion devices with associated drug delivery catheters can facilitate delivery of drug at a higher concentration to a desired treatment site, these devices also meet with limitations. First, the drug delivery catheter may be difficult to position to gain access to the area of the body where drug delivery is desired, e.g., the drug delivery catheter may be limited in its length, or relatively inflexible or otherwise difficult to shape to the tortuous bends in the drug delivery pathway to the treatment site. Second, if the drug delivery catheter is removed or disturbed in order to replenish or replace the drug contained in the infusion device, the entire, tedious procedure for positioning the drug delivery catheter must be repeated.

One method of avoiding constant repositioning of the drug delivery catheter is by having a self-sealable septum associated with the drug reservoir of the infusion device and positioned outside or just under the skin to allow for injection of additional drug into the reservoir (see, e.g., U.S. Pat. Nos. 5,713,858; 5,836,935; 4,816,016; 4,405,305; 5,092,849; 4,929,236; and 5,085,656). However, this method requires the patient be subjected to frequent injections. Furthermore, drug delivery is generally limited to only the region surrounding locations within the body where the infusion device may be implanted, i.e., the device must be implanted so as to allow easy access for injections. Another method of avoiding constant catheter repositioning uses a drug delivery catheter that can be disengaged from the drug delivery device (see, e.g., U.S. Pat. Nos. 5,713,847; 4,692,147; 5,711,316). However, such detachment and reattachment of the drug delivery catheter from the drug delivery device increases the risk of leakage, as well as the risk of contaminants being introduced into the drug delivery pathway.

Still another method for avoiding the repositioning the drug delivery catheter involves a device that is inserted into the subject to maintain a conduit from an external access site to the desired treatment site (see, e.g., U.S. Pat. Nos. 5,792,110; 5,542,923; 5,702,363; 5,053,013; 4,769,005; 5,004, 457; 5,135,525; 4,966,588; 5,257,980; 5,522,803; 4,578,061; 5,464,395; and 4,755,173). However, presently available methods and devices for maintaining such conduits are not completely implantable within the subject, are not suitable for long-term drug delivery, and/or do not provide for delivery of drug to a site deep within the body (e.g., a treatment site other than a subcutaneous or subdermal treatment site). For example, use of such devices is often associated with substantial discomfort or inconvenience to the subject (e.g., due to the use of, for example, a rigid, trocar-like device to maintain the conduit to the treatment site, see, e.g., U.S. Pat. No. 5,792,110), or require the use of equipment that makes such devices and methods impractical for long-term therapy (see, e.g., U.S. Pat. No. 5,004,457). Other presently available devices and methods require the use of a needle, which can cause substantial discomfort to the patient, is generally not suitable for long-term implantation, and thus is generally not suitable for long-term therapy (see, e.g., U.S. Pat. Nos. 5,257,908; 5,522,803; 4,578,061; 5,464,395; 5,464,395; and 4,755,173).

There is a need in the field for a drug delivery system that is completely implantable and provides for convenient, repeated access to a treatment site. The present invention addresses these problems.

### SUMMARY OF THE INVENTION

The present invention provides an implantable guide for access to a treatment site. The implantable guide comprises a proximal end, a distal end, and a guide body defining a lumen, and can optionally comprise a stable positioning element for stably positioning a drug delivery device within the guide. The guide can be provided in connection with a drug delivery device. In use, the guide is implanted within a subject so as to provide a conduit through which a drug delivery device can be retrievably introduced to facilitate delivery of drug to a treatment site within a subject at a site distal to an accessible implantation site. The drug delivery device is then positioned within the guide lumen to provide for delivery of drug from the drug delivery to the desired treatment site.

In one aspect the invention features an implantable guide for facilitating repeated access to a treatment site in a subject, where the guide comprises a proximal end, a distal end, a guide body, and a stable positioning element. The guide body defines a lumen extending from the guide proximal end to the guide distal end, and the stable positioning element facilitates stable positioning at least a portion of a drug delivery device within the guide for delivery of a drug from the drug delivery device and through the guide distal end.

In another aspect, the invention features a system for delivery of drug to a treatment site comprising 1) a flexible guide comprising a proximal end, a distal end, a guide body, and a stable positioning element, where the guide body defines a lumen extending from the guide proximal end to the guide distal end; and 2) a drug delivery device at least a portion of which is removably and stably positioned within the guide lumen. The drug delivery device is positioned for delivery of drug from a drug reservoir of the drug delivery device and through the distal end of the guide lumen. In specific embodiments, the drug delivery device comprises a drug release device comprising a drug reservoir, a distal portion defining a drug delivery orifice, and a drug delivery catheter comprising a drug delivery catheter proximal end and a drug delivery catheter distal end, where the drug delivery catheter proximal end is coupled to the drug release device to provide a drug delivery pathway from the drug reservoir, through the orifice, and through a lumen of the drug delivery catheter to the drug delivery catheter distal end. The drug release device is positioned at the guide proximal end and the drug delivery catheter is positioned within the guide lumen.

In still another aspect the invention features a drug delivery device adapted for retention in a guide of the invention. The drug delivery device comprises a drug release device and a drug delivery catheter. The drug release device distal portion defines an orifice. The drug delivery catheter comprises a proximal end and a distal end, with the proximal end being coupled to the drug release device to provide a drug flow pathway from the reservoir, through the orifice, and through a lumen of the drug delivery catheter.

In another aspect the invention features a method for site-specific drug delivery. The method comprises the steps of implanting a guide into a subject to provide for placement of the guide catheter distal end at a treatment site, and inserting a drug delivery device into the implanted guide so that the drug delivery device is stably positioned at a proximal end of the guide and provides for delivery of drug to a distal end of the guide and to the treatment site.

In another aspect the invention features a method of providing access to a treatment site by implanting a guide of the invention into a subject to provide for placement of the guide distal end at a treatment site, thereby defining a conduit for access to the treatment site.

A primary object of the invention is to provide for a drug delivery system that is completely implantable and allows convenient placement of a drug delivery device and replacement of the drug delivery device without loss of access to the treatment site.

It is another object of the invention to provide a drug delivery system that can be used with a variety of drug release devices to accomplish site-specific drug delivery.

An important advantage of the invention is that the invention facilitates access and re-access of a drug delivery system to the site where drug is desired to be delivered.

Another important advantage of the invention is that the clinician or other user avoids the tedium of re-accessing the treatment site after removal of the drug delivery device and drug delivery catheter.

Another advantage of the invention is that the need for a fluid path coupler, such as that required in detachable drug infusion pump and catheter system, is completely avoided. For example, replacement of the drug delivery device does not require detaching the portion of the drug delivery device housing the drug reservoir from the drug delivery catheter, thus risking contamination of the drug delivery catheter and thus delivery of such contaminants to the treatment site.

Another advantage of the invention is that the drug delivery device can be removed and replaced without coupling and uncoupling the actual drug conduit from the drug release device, thus substantially reducing risk of leakage of drug from the drug release device.

Another advantage is that the drug delivery device can be supplied so that it is primed with drug, e.g., the drug delivery catheter of the device is substantially filled with drug, thus reducing delivery start-up time, i.e., time related to movement of the drug from the drug release device to the distal end of the drug delivery catheter. This feature is particularly advantageous where the dug release device releases drug at relatively low flow rates (e.g., 0.4 μl/day).

Still another advantage of the invention is that the invention can use a material that is relatively more difficult to implant (e.g., a relatively stiff catheter material) for the drug delivery catheter in combination with a guide comprising a material that is relatively easier to implant. Thus, the guide can be designed to facilitate placement of the drug delivery catheter at the treatment site with minimal trauma to the subject, e.g., once in place, the guide protects the subject during placement of the stiffer drug delivery catheter to provide for delivery of drug to the treatment site.

Another advantage of the invention is that the invention can be used in a variety of therapeutic and diagnostic applications. For example, the invention can be used to accomplish controlled delivery of a relatively small amount of drug over a selected period of time (e.g., several hours to several days, weeks, or months) or with delivery of a bolus dose of drug over a relatively short period of time (e.g., a few minutes to hours). The invention can also be used to irrigate a treatment site, e.g., with disinfectant. Alternatively or in addition, the invention can be used as a sampling device, e.g., by inserting a catheter through the guide that is connected to a vacuum source to withdraw fluid and/or tissue from the treatment site to facilitate diagnosis or prognosis of the subject.

Yet another advantage of the invention is that it can be used with any of a variety of drug delivery devices, including those that comprise an externally positioned drug release device or an implanted drug release device. The invention can also be used with drug delivery devices that comprise a drug delivery catheter, which catheter can be composed of a relatively permeable or relatively impermeable material. The invention is also amenable for use with a guide comprising relatively permeable or relatively impermeable material (e.g., a relatively permeable guide can be used with a drug delivery device having a relatively impermeable drug delivery catheter, and a drug delivery device comprising a relatively permeable drug delivery catheter can be used with a relatively impermeable guide).

These and other objects, advantages and features of the present invention will become apparent to those skilled in the art upon reading this disclosure in combination with drawings wherein like numerals refer to like components throughout.

### BRIEF DESCRIPTION OF THE DRAWINGS

FIG. 1 is a cut-away view showing an exemplary guide 10 of the invention.

FIG. 2 is a cut-away view showing an exemplary drug delivery device 50 of the invention.

FIG. 3 is a cut-away view showing a guide 10 of the invention with a drug delivery device 50 inserted therein.

FIG. 4 is a cut-away view of a multi-lumen guide 10 having multiple drug delivery devices 50 stably positioned within the guide 10.

FIG. 5 is a cross-sectional view of a multi-lumen guide 10 with multiple drug delivery catheters 60 positioned therein.

FIG. 6 is a cross-sectional view of a single lumen guide 10 having two drug delivery catheters 60 positioned therein.

FIGS. 7 and 8 are detailed, cut-away views of the distal end 62 of a drug delivery catheter 60 positioned within a guide 10 with a tapered distal end.

FIGS. 9 and 10 are detailed, cut-away views of the distal end 62 of a drug delivery catheter 60 positioned within a guide 10 according to the invention.

FIGS. 11 and 12 are illustrations of exemplary pre-set shapes for a guide.

FIG. 13 is a cut-away view of an exemplary alternative embodiment of the invention in which the proximal end of the guide is formed into a guide chamber 16, the distal end of which forms a partial cap over the proximal end of the drug delivery device and retains the drug delivery device within the guide 10.

FIGS. 14 and 15 are detailed, cut-away views of the proximal end 11 of the guide showing various embodiments of the stable positioning element, which serves to stably position the drug delivery device within the guide 10.

FIG. 16. is a detailed, cut-away view of an alternative embodiment of the proximal end of a guide 10 of the invention.

FIG. 17 is a cut-away view of an exemplary alternative embodiment of the invention, where the proximal end of the guide provides a stable positioning element, and is associated with a distal portion of the drug release device.

FIG. 18 is a cut-away view of an exemplary alternative embodiment of the invention, where the proximal end of the guide provides a stable positioning element, and is associated with a distal portion of the drug release device.

FIG. 19 is a cut-away view of a guide 10 showing stable positioning of a drug delivery device within the guide 10 by removably attaching the guide proximal end 11 to a distal portion of a drug release device 70.

FIG. 20 is a perspective view of a drug delivery device 50 comprising a mechanical or electromechanical pump 75 as a drug release device (phantom-lined), where the drug delivery device is positioned for use within a guide 10.

FIG. 21 is an exploded view of FIG. 19 showing stable positioning of a drug delivery device by attachment of a drug release device 70 into a guide 10 using a snap-fit configuration.

FIG. 22 is a cut-away view of an exemplary alternative embodiment of the invention, illustrating the attachment of the drug delivery device to the guide by means of snap fit tab(s) of the drug delivery device inserted into snap fit recesses of the guide.

FIG. 23 is a cut-away view of an alternative embodiment for stable positioning of the drug delivery device 70 by attachment of a drug release device 70 into a guide 10 using a threaded male member 94.

FIG. 24 is an exploded view of a stable positioning element that stably positions the drug delivery device within a guide 10 by means of a threaded luer coupling member 96 that is threaded on to a threaded male portion 97 of a drug delivery device 70.

FIG. 25 is a cut-away view of a guide 10 having a self-sealing barrier element 25 positioned within a guide chamber 16.

FIG. 26 is a cut-away view of an exemplary drug delivery device 50 suitable for use with a guide of FIG. 25.

FIG. 27 is a cut-away view of an alternative embodiment of the drug delivery device illustrated in FIG. 26.

FIG. 28 is a cut-away view of an exemplary drug delivery device.

FIG. 29 is a cut-away view illustrating placement of a guide 10 using a tunneling device 85.

FIG. 30 is a cut-away view illustrating placement of a guide 10 into a tunneling device 85 using a wire 87 as reinforcement for pushing the guide 10 through the tunneling device 85.

FIG. 31 is a cut-away view illustrating a guide 10 comprising a reinforcing element channel 24 through which a wire 87 is introduced to facilitate placement of the guide.

FIG. 32 is a cross-section of the guide 10 and reinforcing element channel of FIG. 31.

FIG. 33 is a cut-away view of a drug delivery device 50 positioned within a guide 10, where the guide comprises sealing elements 28.

FIGS. 34 and 35 are perspective and cut-away views, respectively, of a drug delivery device 50 positioned within a guide 10, where the stable positioning element is provided as a snap fit tab 92 positioned in a wall of the guide 10, which snap fit tab 92 is seated within a snap fit tab recess 93 on the outer wall of the drug delivery device 50.

FIG. 36 is a perspective view of a drug delivery device 50 positioned within a guide 10, where the stable positioning element is provided as a luer lock composed of a tab 99 locked within a tab receiving slot 100.

FIG. 37 is a cut-away view of a guide 10 having a proximal end adapted for attachment to a guide chamber 16, where the guide is positioned within a insertion cannula 85.

FIG. 38 is a cut-away view of a guide 10 having a proximal end adapted for attachment to a guide chamber 16 and a guide chamber 16 attached by means of an attaching element 30.

FIG. 39 is cut-away view of a guide having a reinforcing element channel 24 with a closed distal end with a mandrel 110 positioned within the channel 24.

FIG. 40 is a cut-away view of a guide 10 having a reinforcing element channel 24 with a closed distal end, with a drug delivery device 50 positioned within the guide.

### DETAILED DESCRIPTION OF THE PREFERRED EMBODIMENTS

Before the present drug delivery system, method of drug delivery, and specific devices and formulations used in connection with such are described, it is to be understood that this invention is not limited to the particular embodiments described, as such methods, devices, and formulations may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural referents unless the context clearly dictates otherwise. Thus, for example, reference to “a formulation” includes mixtures of different formulations, and reference to “the method of delivery” includes reference to equivalent steps and methods known to those skilled in the art, and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the specific methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention.

Further, the dates of publication provided may be different from the actual publication dates which may need to be independently confirmed.

Definitions

“Implantable” encompasses, but is not necessarily limited to, devices that can be substantially completely implanted within the body of a subject. For example, an “implantable” device that is substantially completely implantable is one that is implanted at a subcutaneous site and, in some embodiments, extends to a site distal to the subcutaneous site (e.g., to a treatment site located deeper within the subject's body).

“Controlled release” as used herein (e.g., in the context of “controlled drug release”) is meant to encompass release of substance (e.g., a drug) at a selected or otherwise controllable rate, interval, and/or amount. “Controlled release” thus encompasses, but is not necessarily limited to, substantially continuous delivery, patterned delivery (e.g., intermittent delivery over a period of time that is interrupted by regular or irregular time intervals), and delivery of a bolus of a selected substance (e.g., as a pre-determined, discrete amount of a substance, over a relatively short period of time (e.g., a few seconds or minutes).

The term “controlled drug release device” is meant to encompass any device that provides for controlled release of a drug or other desired substance and that can be adapted for use in the drug delivery device of the invention, e.g., a drug delivery device that provides for controlled release of drug through a drug delivery catheter associated with the drug reservoir, and at a rate that is suitable to accomplish delivery of a therapeutically effective amount of drug to a treatment site according to the methods of the invention.

The term “treatment site” as used herein is meant to refer to a desired site for delivery of drug from a drug delivery device of the invention, and/or a site from which sampling is desired, e.g., for diagnosis and/or prognosis. “Treatment site” is thus meant to include, although is not necessarily limited to, a subcutaneous, intravenous, intrathecal, intraorbital, intraocular, intraaural, intratympanic, intramuscular, intra-arterial, intra-articular, intracavitary, intraductal, intraglandular, intravascular, intranasal, intraperitoneal, intraspinal, epidural, intracranial, intracardial, intrapericardial, peritumoral, or intratumoral (i.e., within a cancerous growth) site within a subject. “Treatment site” thus also encompasses intracavitary sites, e.g., sites within or near a selected organ or tissue (e.g., central nervous system (e.g., spinal fluid), kidney, liver, pancreas, heart (e.g., intrapericardial), lung, eye, inner ear, middle ear, cochlea, lymph nodes, breast, prostate, ovaries, testicles, thyroid, spleen, etc.), into arteries that feed a selected organ to tissue, or at a site associated with a microbial infection (e.g., bacterial, viral, parasitic or fungal infection).

The term “access site” or “implantation site” is used to refer to a site on or in a subject at which a guide and drug delivery device of the invention are introduced for implantation and positioning within the subject's body, e.g., for delivery of drug to a desired treatment site. For example, where a guide is implanted in a subject for delivery of drug to the spinal cord, the access site or implantation site can be a subcutaneous site at which a proximal end of the guide is substantially retained, and the treatment site is a position within or adjacent the spinal cord (treatment site) at which a distal end of the guide is positioned for delivery of drug.

“Drug delivery system” as used herein is meant to refer to a combination of a guide and drug delivery device of the invention suitable for use in delivery of a drug to a treatment site.

The term “subject” is meant any subject, generally a mammal (e.g., human, canine, feline, equine, bovine, etc.), to which drug delivery is desired.

The term “impermeable” with reference to a dispensing device means that the material is sufficiently impermeable to environmental fluids as well as ingredients contained within the dispensing device such that the migration of such materials into or out of the device through the impermeable device is so low as to have substantially no adverse impact on the function of the device during the delivery period.

The term “semipermeable” means that the material is selectively permeable, e.g., permeable to external fluids but substantially impermeable to other ingredients contained within the dispensing device and the environment of use.

The term “drug” as used herein is meant to encompass any substance suitable for delivery to a treatment site of a subject, which substances can include pharmaceutically active drugs, as well as biocompatible substances that do not exhibit a pharmaceutical activity in and of themselves, but that provide for a desired effect at a treatment site, e.g., to flush or irrigate a treatment site (e.g., saline).

“Pharmaceutically active drug,” “therapeutic agent,” “therapeutic drug,” and the like are used interchangeably herein to refer to any chemical compound which, when provided to a subject, facilitates a therapeutic effect. Such drugs may optionally be provided in combination with pharmaceutically acceptable carriers and/or other additional compositions such as antioxidants, stable positioning agents, permeation enhancers, etc. Drugs compatible for delivery using the devices and methods of the invention are discussed below, and are readily apparent to the ordinarily skilled artisan upon reading the disclosure provided herein.

The term “therapeutically effective amount” is meant an amount of a therapeutic agent, or a rate of delivery of a therapeutic agent, effective to facilitate a desired therapeutic effect. The precise desired therapeutic effect will vary according to the condition to be treated, the drug to be administered, and a variety of other factors that are appreciated by those of ordinary skill in the art. Determinations of precise dosages are routine and well within the skill in the art.

The term “treatment” is used here to cover any treatment of any disease or condition in a mammal, particularly a human, and includes: a) preventing a disease, condition, or symptom of a disease or condition from occurring in a subject which may be predisposed to the disease but has not yet been diagnosed as having it; b) inhibiting a disease, condition, or symptom of a disease or condition, e.g., arresting its development and/or delaying its onset or manifestation in the patient; and/or c) relieving a disease, condition, or symptom of a disease or condition, e.g., causing regression of the disease and/or its symptoms.

Overview of the Invention

The present invention, as it relates to an implantable drug delivery system, generally features: 1) an implantable guide comprising a proximal end, a distal end, and a wall defining a lumen; and 2) a drug delivery device minimally comprising a drug release device that can be implantable within the body or left external from the body and, in a preferred embodiment, further comprises a drug delivery catheter. The guide can be substantially permanently implanted within a subject to provide a conduit to a desired treatment site (e.g., a body tissue, organ, or other site). The drug delivery device is then stably positioned within the guide to provide for delivery of drug from the drug delivery device to the treatment site. Preferably, the drug delivery device is retrievable or replaceable, e.g., the drug delivery device can be removed from the guide and, where desirable, another drug delivery device inserted in its place, and can be completely or partially implanted, or completely or partially external to the subject.

In one embodiment, the drug delivery device comprises a drug delivery catheter, which provides for delivery of drug from a drug reservoir to the treatment site. In this embodiment, the drug delivery catheter of the drug delivery device is threaded through the guide so that the drug delivery outlet of the drug delivery catheter is positioned for delivery of drug at the treatment site. At least a portion of the drug delivery device is retained at an accessible access site (e.g., the drug release device portion of the drug delivery device is retained at a subcutaneous access site). In another embodiment, the drug delivery device comprises a leash for retrieving the drug delivery device. Drug delivery devices comprising a leash can be positioned at any point within the lumen of a guide (e.g., at a site any distance from a subcutaneous access site at which the drug delivery device is initially introduced into the guide). In this latter embodiment, the drug delivery device can further comprise a drug delivery catheter, although such may not be necessary.

After delivery of the drug from the drug delivery device is complete (e.g., the drug reservoir is substantially empty) or it is otherwise desirable to terminate delivery of drug, the drug delivery device can be removed from the guide without losing access to the treatment site, i.e., access to the treatment site is maintained by the guide. The guide thus facilitates removal and replacement of the drug delivery device through the same conduit or treatment site access route, without the need to re-establish access the treatment site. Furthermore, the guide and drug delivery device system of the invention allows for exchange and replacement of the drug delivery device without the need to uncouple the drug release device from a drug delivery catheter, thus substantially reducing both the risk of leakage of drug from the device and the risk of contamination of the treatment site (e.g., by introduction of contaminants into the drug delivery catheter). In addition, the drug delivery catheter of the drug delivery device can be coated with silver or otherwise coated or treated with antimicrobial agents, thus further reducing the risk of infection at the treatment site. The entire drug delivery system can be implanted within the subject, and can be provided in a size and configuration that minimizes discomfort or inconvenience to the subject. In addition, all or a portion of the drug delivery device can be retained outside the subject with the drug delivery catheter residing in the guide.

The specific components of the guide and exemplary drug delivery device suitable for use with the guide will now be described in further detail and in relation to the drawings provided herein. The specific components and embodiments of the invention provided below are not meant to be limiting, but rather only illustrative of the claimed invention. For example, while the drug delivery device is illustrated as comprising a controlled drug release device that is an elongate cylinder, and/or the guide is generally illustrated as comprising a stable positioning element that stably positions a drug delivery device within the guide, (e.g., a guide chamber that is of a shape suitable for receiving a drug release device), other forms and types of controlled drug release devices, as well as other forms and variations of the guide, are suitable for use in invention. Moreover, while an osmotic pump is a preferred form of controlled drug release device, other controlled drug release devices are also suitable for use in the drug delivery device and guide of the invention, and thus are contemplated by and within the scope of the present invention.

Guide

In general, the guide comprises a proximal end, a distal end, and a body defining at least one lumen. Typically the guide is provided as an elongated, substantially hollow tube. In one embodiment, the guide further comprises a stabilizing element, which facilitates retention and/or positioning of all or at least a portion of a drug delivery device within the guide, e.g., as during use in drug delivery to a treatment site. FIG. 1 illustrates an exemplary embodiment of the guide 10 of the invention, in which the guide is provided in a substantially hollow, cylinder-type configuration comprising a guide body 20 defining a guide lumen 13, and a stable positioning element, which in this embodiment is exemplified by a guide chamber 16. The guide chamber 16, as well as further exemplary stable positioning elements, are described in more detail below. In the embodiment provided in FIG. 1, the guide chamber 16 is designed to receive and, preferably, retain a drug delivery device 50, generally through reversible association of a drug release device 70 and the guide chamber 16. The guide body 10 further comprises distal and proximal ends 21 and 22. The distal end of the guide chamber 16 defines an opening 17 that is in communication with the lumen of the guide body 20. As illustrated in FIG. 3, the guide is suitable for use with a drug delivery device 50, which in this example comprises a drug delivery catheter 60 threaded through the guide chamber 16, through opening 17, and into the lumen of the guide body 20.

The guide need not comprise a septum or other element that substantially covers the proximal end 11 of the guide when the guide is used in conjunction with a drug delivery device, since access to the guide lumen from the guide proximal end will generally be inhibited or substantially unavailable when a drug delivery device is positioned within the guide (e.g., due to the communication of the drug delivery device, the guide, and a sealing element positioned between an outer wall of the drug delivery device and an inner wall of the guide. Where it is desirable to leave the guide implanted without a drug delivery device in position within the guide, it may be desirable to cap or otherwise temporarily or reversibly close the open proximal end of the guide, e.g., to prevent accumulation of fluids or other biomaterial in the guide and/or to inhibit tissue growth into or within the guide.

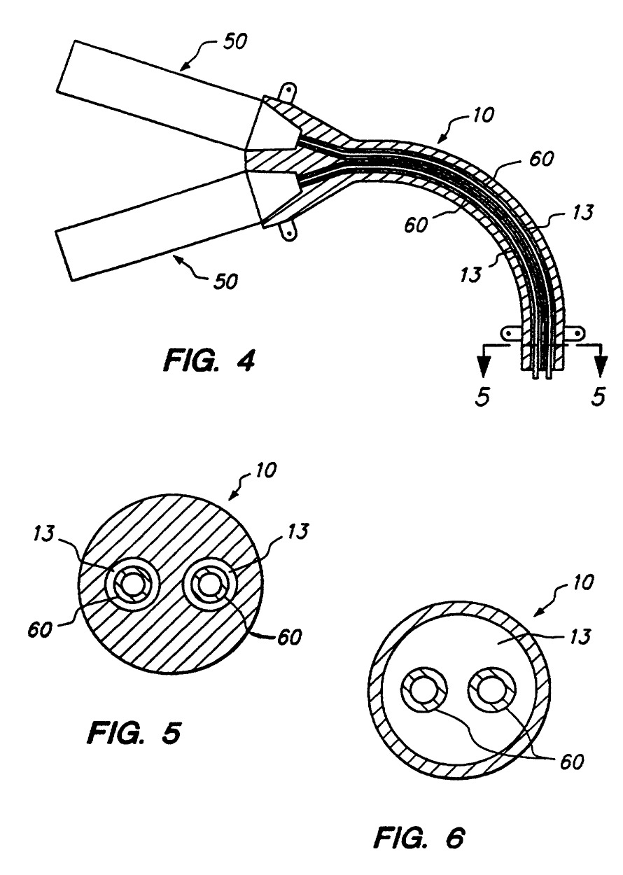

The guide of the invention can be designed for use with a single drug delivery device, or can be designed for use with a plurality (e.g., two or more) drug delivery devices. FIG. 4 illustrates an exemplary guide 10 that is designed for use with two drug delivery devices 50. The drug delivery devices 50 can be stably positioned for use in the guide 10 by any suitable means (e.g., press-fit lock, threaded element, bayonet connector, etc.). The guide 10 can have a plurality of lumen 13 wherein a drug delivery catheter 60 can be positioned within at least one of the lumen (see, e.g., FIG. 5). Alternatively, the guide 10 has a single lumen 13 into which a plurality of drug delivery catheters 60 are introduced (see, e.g., FIG. 6).

The guide 10 can be made of any suitable biocompatible material. Exemplary materials include, but are not necessarily limited to, polymers; metals; glasses; polyolefins (high density polyethylene (HDPE), low density polyethylene (LDPE), linear low density polyethylene (LLDPE), polypropylene (PP), and the like); nylons; polyethylene terephtholate; silicones; urethanes; liquid crystal polymers; PEBAX™; HYTREL™; TEFLON™; perflouroethylene (PFE) perflouroalkoxy resins (PFA); poly(methyl methacrylate) (PMMA); multilaminates of polymer, metals, and/or glass; and the like. The guide can be made of the same materials throughout its length, or may vary in composition over its length.

The guide may comprise a reinforcement element(s) to provide for enhanced stiffness, to avoiding kinking of the guide body, etc. The reinforcement element(s) can be, for example, a coil or braid that is on the outer surface of the guide body, within a wall of the guide body, or positioned on the inner wall of the guide body. The guide 10, as well as other guide components (e.g., a stable positioning element for stably positioning a drug delivery device within the guide), can be made of the same or different materials, and may be manufactured as a single piece (e.g., by molding) or as separate pieces that are subsequently attached one to another using any suitable attachment means.

The material(s) of the guide 10, and particularly of the guide body 20, are generally selected so that the guide is sufficiently flexible to facilitate insertion and placement at the treatment site. The guide can be of substantially the same degree of flexibility or stiffness throughout its length, or may vary in flexibility or stiffness over its length (e.g., a distal portion of the guide body may be more or less flexible than a proximal portion of the guide body). The desired flexibility or stiffness of the guide can be varied with the particular treatment site and/or drug delivery pathway with which the guide is to be used. For example, where the drug delivery pathway is defined in whole or in part by one or a series of biologically defined lumen (e.g., vein artery, capillary, lymphatic, organ duct (e.g., duct of a secretory gland (e.g., salivary gland, liver, pancreas, etc.)), and the like), it may be desirable to use a guide having flexibility sufficient to conform the guide to the biological pathway, e.g., the guide is flexible enough to be deflected by the walls of the biologically defined lumen into which it is introduced. Alternatively, where the drug delivery pathway may be defined as a site deep within a tissue, it may be desirable to select a guide having at least a relatively stiff portion to facilitate placement of the guide. However, the use of relatively stiff guides can be avoided where a tool such as a trocar, guidewire, or other device is used to facilitate implantation.

The dimensions of the guide 10, particularly the guide body 20 and any other elements or components of the guide, can be varied depending upon a variety of factors, such as the particular treatment site to which drug delivery is desired, the access route used to reach the desired treatment site, the dimensions of the components of the drug delivery device (e.g., the dimensions of the drug release device or drug delivery catheter) to be used with the guide, etc. For example, in the exemplary embodiment depicted in FIG. 1, the inside diameter of the guide chamber 16 and of the guide body 20 will generally be sufficiently greater than the outside diameter of the drug release device and drug delivery catheter, respectively, so that the drug delivery device can be reversibly threaded into the guide and retained within the guide while implanted.

In general, the guide body 20 will typically have a length in the range from about 1 cm to 150 cm, usually having a length in the range from about 2-5 cm up to about 50 cm. The outside diameter of the guide body that defines the lumen in which the drug delivery device resides will typically be in the range from about 0.1 mm (0.3 F) or 0.15 mm and up to about 2 mm (6 F) or 2.5-3 mm, usually being in the range from about 0.125 mm (0.4 F) to about 1 mm (3 F). In one embodiment, the guide an outer diameter of about 0.020″. The inner diameter of the guide is generally in the range from about 0.025 mm-0.03 mm to about 1.5-2 mm, usually being in the range from about 0.05 mm to 1 mm. Normally guides comprising a guide body having larger outside diameters usually having larger lumen diameters. In one embodiment, the guide body has an inner diameter of about 0.035″.

In general, the guide body length ranges from about 1 cm to about 200 cm, usually from about 15 cm to about 40 cm; an outside diameter in the range from about 0.125 mm (0.4 F) to about 3 mm, usually from about 0.66 mm (2 F) to about 0.5 mm; and an inside diameter in the range from about 0.05 mm to about to about 2 mm, usually from about 0.075 mm to about 0.5 mm (2 F). The inside diameter of the guide body is generally greater than the outside diameter of a drug delivery catheter and/or drug delivery device that is to be used in conjunction with the guide.

The dimensions of the guide can vary according to a variety of factors, e.g., the dimensions of the drug delivery device with which the guide is to be used, the treatment site, the material of the guide, etc. For example, where the guide body is comprised of a material that has elastic qualities, the guide body may have an inside diameter that is smaller than or equal to the outside diameter of the drug delivery device or a position thereof. Upon insertion of the drug delivery device, the guide expands or stretches to accommodate insertion of the drug delivery device into the guide lumen.

In addition to the uses described herein in drug delivery, the guide of the invention can also be used as a conduit for other purposes. For example, the guide can be used in conjunction with a sampling device, e.g., a vacuum source can be attached to a catheter, which is threaded through a lumen of a guide to the treatment site. The sampling device positioned within a guide of the invention can be used to extract a biological sample (e.g., biological fluids (e.g., blood, spinal fluid, lymph, etc.), cells, tissue, etc.) from a treatment site. Other uses of the guide, drug delivery device, and drug delivery system of the invention will be readily apparent to the ordinarily skilled artisan upon reading of the disclosure provided herein, and as such are contemplated and encompassed by the present invention.

Exemplary embodiments of guides contemplated and within the scope of the invention are described in more detail below.

Guide Body

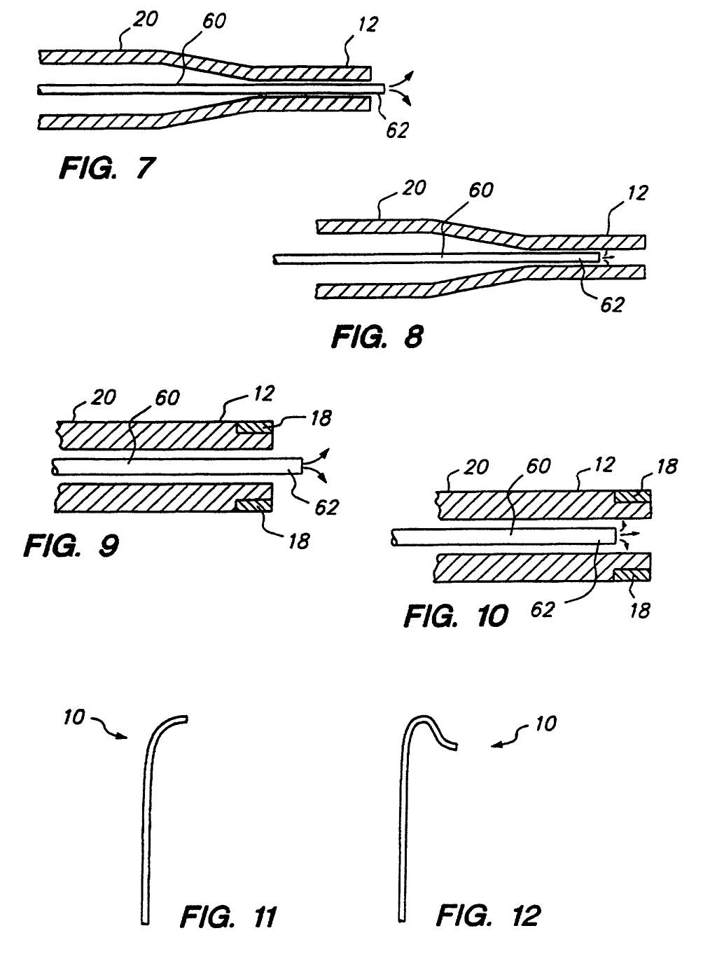

In one embodiment, the body 20 of the guide 10 is provided as a substantially hollow tube. The guide body 20 is designed so as to facilitate the placement of the drug delivery catheter 60 of a drug delivery device 50 of the invention through the conduit created by the lumen of the guide body 20, thus providing for delivery of drug from the distal end 61 of the drug delivery catheter 60 to the treatment site. The distal end 12 of the guide 10 can be provided in any of a variety of configurations. For example, the distal end 12 may be provided in a closed configuration, such that the inner diameter of the distal end 12 is less than the diameter of the proximal portion of the guide body 20, but greater than the outer diameter of the distal end 62 of the drug delivery catheter 60 (FIG. 7).

In one embodiment, the distal end 12 of the guide body 20 comprises a valve, e.g., a duckbill valve, that is forced open upon insertion of the drug delivery catheter distal end 62 into the guide distal end 12 (FIG. 8). In this “closed” configuration, drug may be delivered from the drug delivery catheter distal end through the tip of the guide distal end 12, thus avoiding insertion of drug delivery catheter directly into the treatment site. This latter embodiment may be particularly advantageous where, for example, the drug delivery catheter is made of a relatively stiff material and/or may have a sharp end that may damage tissue at the treatment site. This embodiment may also be particularly advantageous where the treatment site is particularly sensitive. Moreover, providing a valve at the guide distal end 12 can help the clinician or other operator implanting the device from inserting a drug delivery device having a drug delivery catheter that is too long for use with the guide implanted in the patient, thus avoiding insertion of the drug delivery catheter into tissue beyond the distal end of the guide.

Where desired, the length of the drug delivery catheter 60 relative to the length of the guide body 20 may be varied. For example, the catheter 60 can be of a length such that the distal end 62 of the catheter 60 is at a point beyond the distal end 12 of the guide 10 (FIGS. 7 and 9) or at a point within the distal end 12 of the guide 10 (FIGS. 8 and 10). When the guide distal end 12 is provided as the closed distal end embodiment as depicted in FIG. 8 and the catheter distal end 62 is seated within the guide distal end 12, drug is delivered through the catheter 60 and out of the tip of the guide 10. Where the guide distal end 12 is provided as the open distal end embodiment as depicted in FIG. 10 and the catheter distal end 62 is seated within the guide distal end 12, drug may diffuse in all directions within the guide lumen, including out the guide distal end 12.

The guide 10 can be modified as may be suitable for particular uses, e.g., as may be required or optimal for use for drug delivery to various treatment sites. For example, the guide can comprise coatings such as hydrophilic, anti-thrombogenic, low-friction, or hydrophobic coatings, which can be placed over the inner or outer surface of the guide body. Additionally, the distal end of the guide can be formed into a desired geometry, as described above, and the strength and flexibility characteristics of the guide body can be further modified by varying the materials used in the manufacture of the guide. For example, the guide can be multi-laminate with a biocompatible outer surface and a lubricated lining. As described for the drug delivery catheter, the guide can be formed into a pre-set shape or geometry to facilitate insertion and/or drug delivery to at desired treatment site.

The guide can be made from a material or matrix of materials (e.g., reinforced construction with braided wire, coiled, wire, etc.) or can be formed from multiple layers of materials. The guide body can be formed into any of a variety of pre-set shapes, which may be particularly desirable to facilitate access to a particular treatment site. For example, particular pre-set shapes are useful to facilitate delivery of drug through a coronary artery of the right or left side of the heart. Furthermore, particular guide shapes may be desired for use in drug delivery to treatment sites such as the spine, inner ear, pericardial space, or a location within an organ (e.g., to delivery drug to a tumor of a selected organ). Exemplary pre-set guide body shapes useful in delivery of drug to via a coronary artery include, but are not limited to, those shown in FIG. 11 (hockey stick) and FIG. 12 (amplatz shape).

The guide 10 can be further modified by providing radiopaque markers 18 at one or more locations along its length. In one embodiment, radiopaque markers are provided at the tip of the guide distal end (FIGS. 9 and 10). Such radiopaque markers can comprise metal rings (e.g., platinum, palladium, gold, etc.), or can be defined by impregnating the body of the guide with appropriate radiopaque dyes or other radiopaque materials. The provision of radiopaque markers is well known in the art.

Positioning and/or Retention of a Drug Delivery Device within a Guide

In one embodiment, the guide comprises a stable positioning element. The stable positioning element is any element that facilitates association or coupling of a drug delivery device with a guide, e.g., as during use in drug delivery to a treatment site. For example, where the drug delivery device does not comprise a drug delivery catheter, the stable positioning element stably positions all or a substantial portion of the drug release device of the drug delivery device within the guide lumen. Where the drug delivery device comprises a drug delivery catheter, the stable positioning element stably positions at least a portion of the drug delivery catheter within the guide, and further preferably stably retains or positions the drug release device of the drug delivery device immediately adjacent the guide proximal end (i.e., such that the drug release device communicates with at least a portion of the guide proximal end) or stably retains or positions substantially all or a portion of the drug release device within the guide lumen. Any of a variety of such means are compatible for use in the drug delivery system of the invention. Non-limiting examples of such means are provided below.

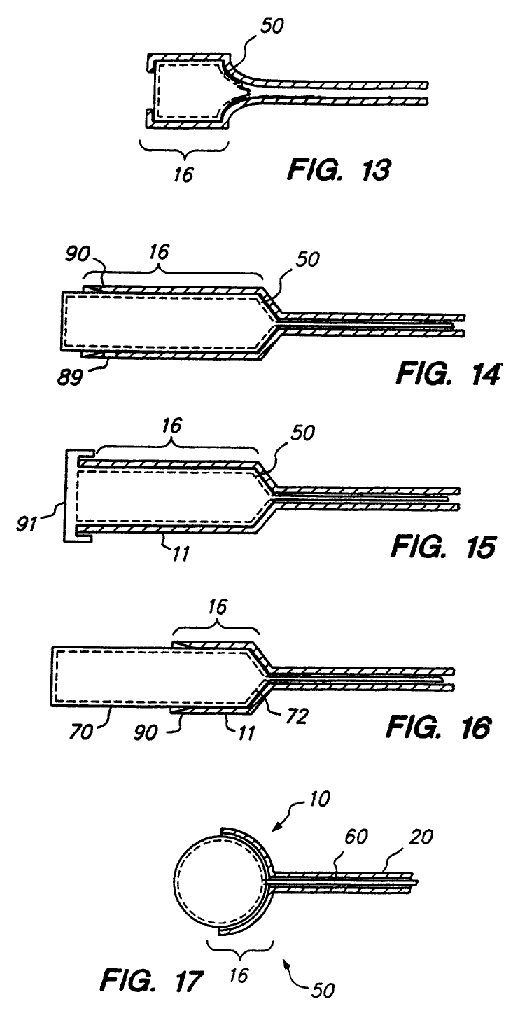

In one exemplary embodiment, illustrated in FIGS. 1 and 3, the guide 10 comprises a guide chamber 16 as the stable positioning element. In this embodiment, the guide chamber 16 of guide 10 is designed for receiving and positioning the drug release device 70 of the drug delivery device 50. To this end, the guide chamber 16 and/or the drug delivery device that is to be positioned within the guide chamber 16 can be designed to facilitate retention of the drug delivery device within the guide chamber 16. The walls of the guide chamber 16 can completely encompass the drug release device of the drug delivery device (as exemplified in FIG. 3), or can be of any length sufficient to accomplish stable positioning, and preferably retention, of the drug delivery device within the guide so that drug is delivered from the drug delivery device to the treatment site. The guide chamber 16 can comprise additional elements to accomplish retention of the drug delivery device within the guide, such as an end cap portion that is permanently or removably attached to a distal end of the guide, and which can cover the proximal end of the guide (see, e.g., FIG. 13).

In another example, the stable positioning element is provided as a “locking/docking” mechanism. Examples of such locking/docking mechanisms that can serve as are provided in FIGS. 14-16. In one embodiment, the proximal end 11 of the guide 10 is in the form of a press-fit lock 90, so that upon insertion of the drug delivery device 50 into the guide 10, the body of the drug release device 70 of the drug delivery device 50 is held in place by force of the walls of the guide chamber 16 (FIG. 14). Preferably, a vent 89 is provided to allow escape of any fluid within the guide chamber 16 upon pressing the drug release device 70 into place.

Alternatively, the drug delivery device comprises a stable positioning element that can interact with, for example, a proximal end of the guide. For example, as illustrated in FIG. 15, a distal portion of the drug delivery device 50 forms a flanged end cap portion 91 that, when the drug delivery device 50 is seated within the guide 10, overlays the guide proximal end 11 and retains the drug delivery device 50 within the guide 10. In this latter embodiment, the proximal end of the guide can be fashioned from compressible material, so that the proximal end can be depressed, the drug delivery device with a flanged-end cap portion positioned within the guide, and the proximal end released so that the wall of the distal end presses against the inner side of the flanged end cap of the drug delivery device.

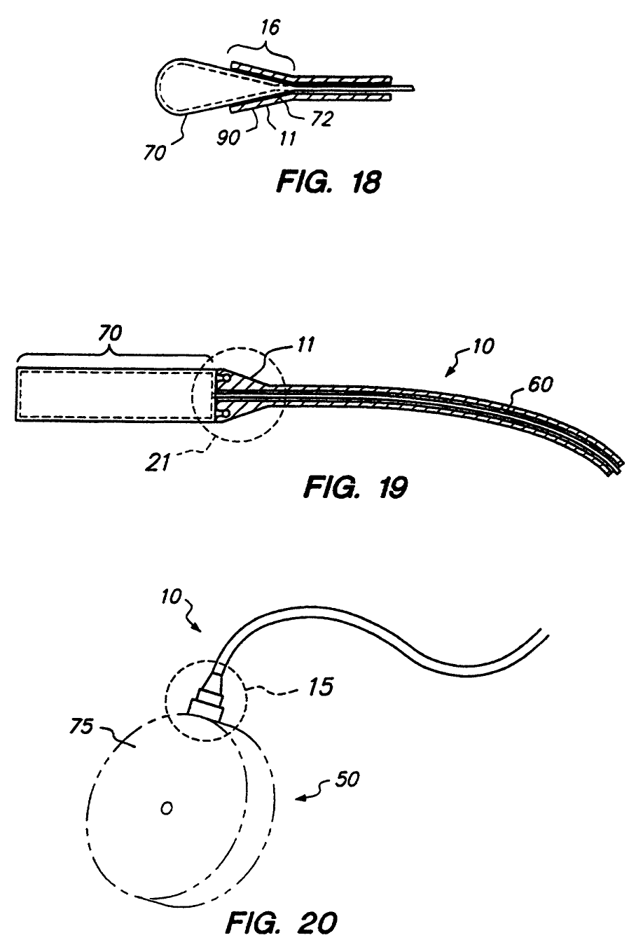

In another embodiment, the locking/docking mechanism is provided by attachment of the guide proximal end 11 to a distal portion of the drug delivery device 50, e.g., by means of a press fit lock 90 (see, e.g., FIGS. 16, 17, and 18). Other exemplary locking/docking mechanisms suitable for use in the invention include, but are not necessarily limited to, bayonet style connectors, thread connectors (e.g., where the proximal end of the release device is provided with a threaded cap that overlays and threads onto a threaded portion of the guide distal end), and various retaining means known in the art.

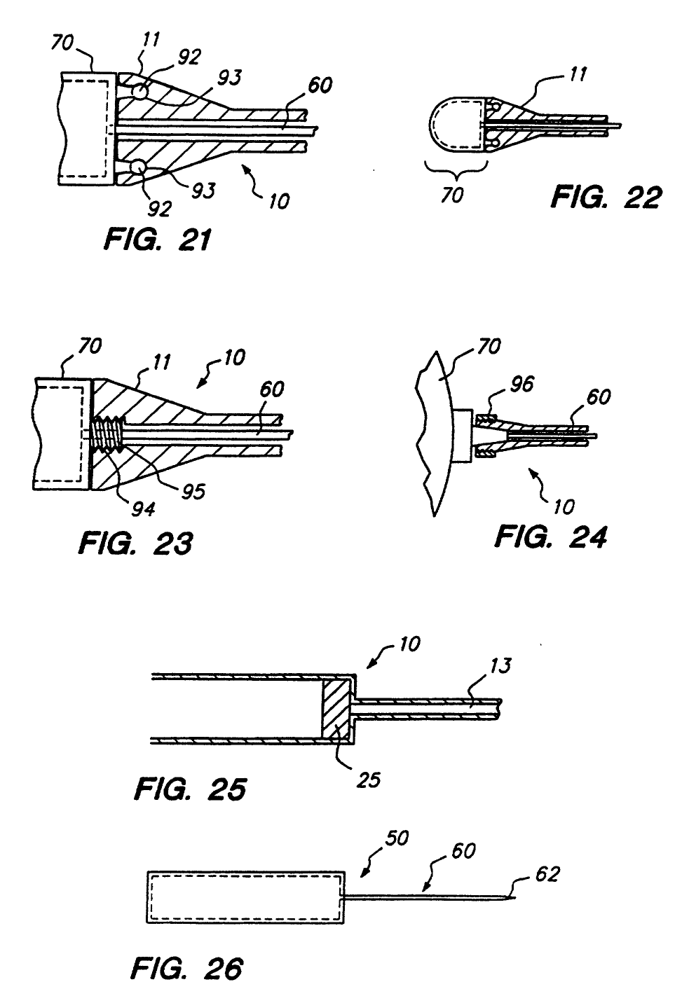

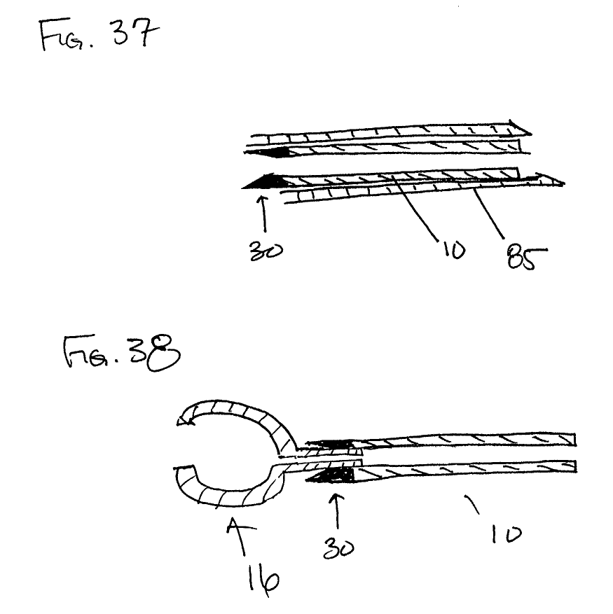

Alternatively, the stable positioning element is designed from a proximal end of the guide to provide for association of the guide with a distal portion of a drug release device of the drug delivery device. For example, as illustrated in FIGS. 19 and 20, the proximal end 11 of the guide 10 can be fashioned so as to be removably attached to a distal end portion of a drug release device 70. The drug release device 70 can be secured within the guide proximal end by means of insertion of a snap fit tab 92 into a snap fit recess 93. For example, the snap fit tab 92 portion can be positioned at the distal end of the drug release device 70 and mate with a snap fit recess 93 at a proximal end 11 of the guide 10 (see, e.g., FIGS. 21 and 22). In one preferred embodiment, the snap fit tab 92 portion is positioned on the guide (e.g., as a portion of a guide chamber) and mates with a snap fit recess 93 on the outer surface of the drug release device 70 of the drug delivery device 50 (see, e.g., FIGS. 34 and 35). The snap fit recess 93 can be fashioned as a circumferential recess around the outer diameter of a portion of the drug release device 70. Alternatively, a threaded male member 94 can be provided at the distal end of the release device 70 and threaded into a threaded recess 95 within the proximal end 11 of the guide 10 (see FIG. 23).

In another embodiment, the proximal end 11 of the guide 10 is provided with a threaded luer coupling member 96 (exemplified by a female luer lock) that is threaded on to a threaded male portion 97 of a drug delivery device 70 (see, e.g., FIG. 24). Preferably, the threaded coupling member 96 can be threaded onto the threaded male portion 97 by manipulation of substantially only the threaded coupling member 96, thus avoiding further manipulation of the drug delivery device 50. In one preferred embodiment, the lock is provided as a bayonet style connector provided as a tab 99 positioned on a proximal portion of the drug delivery device 50, where the tab 99 is received by a tab receiving slot 100 positioned at a proximal end of the guide 10 (see, e.g., FIG. 36).

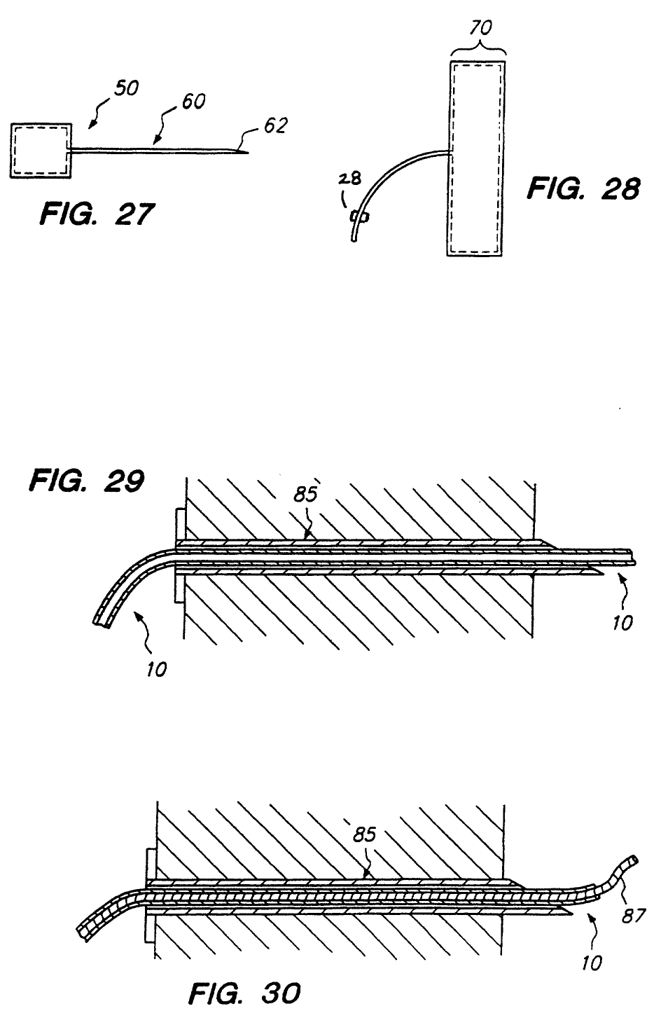

In still another embodiment, the guide 10 comprises a self-sealing barrier element 25 positioned at a proximal end of the guide 10 (see, e.g., FIG. 25). The self-sealing barrier element 25 may be cross-linked by a hydrophobic polymer. In use, a drug delivery device 50 comprising a drug delivery catheter 60 having a relatively sharp distal end 62 (see, e.g., FIGS. 26 and 27) is inserted into the guide 10 so that the sharp distal end 62 pierces the self-sealing barrier element 25. The drug delivery device 50 is stably positioned within the guide by virtue of the self-sealing barrier element 25, which also provides for isolation of at least a portion of the guide lumen 13 from the environment during implantation of the drug delivery device 50. As such the self-sealing barrier element 25 must be of a thickness sufficient to inhibit movement of the drug delivery device within and/or out of the guide lumen.

Alternatively or in addition, the drug delivery device and/or guide can be anchored at an external or internal site with respect to the subject by any suitable conventional means. For example, sutures can be used to secure the drug delivery device proximal end at or near an implantation site. The guide can be similarly be anchored within the subject.

Sealing Elements

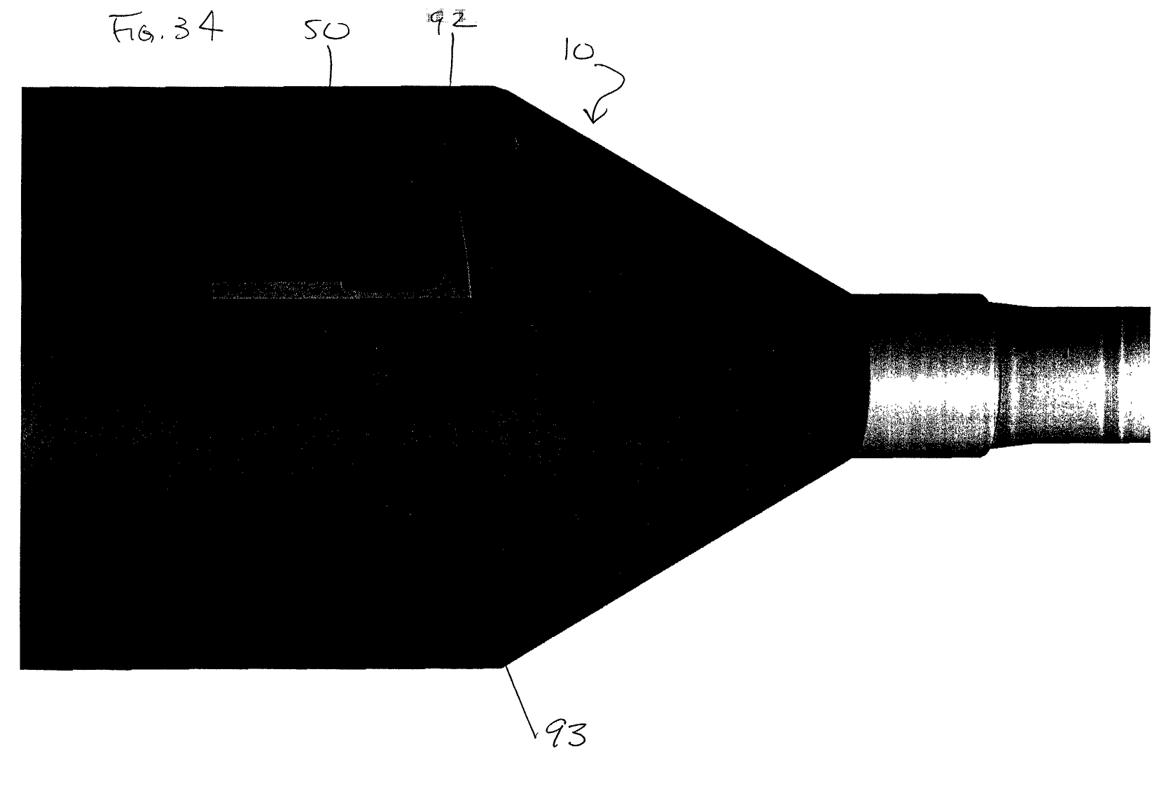

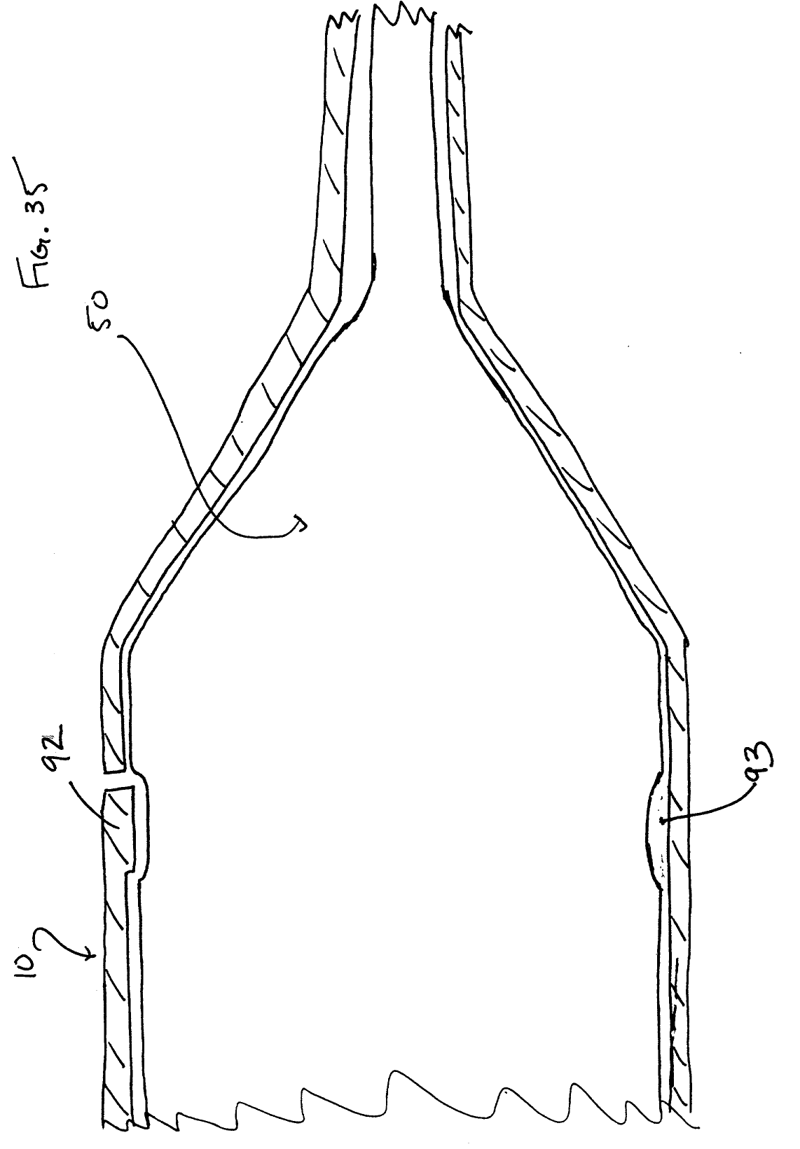

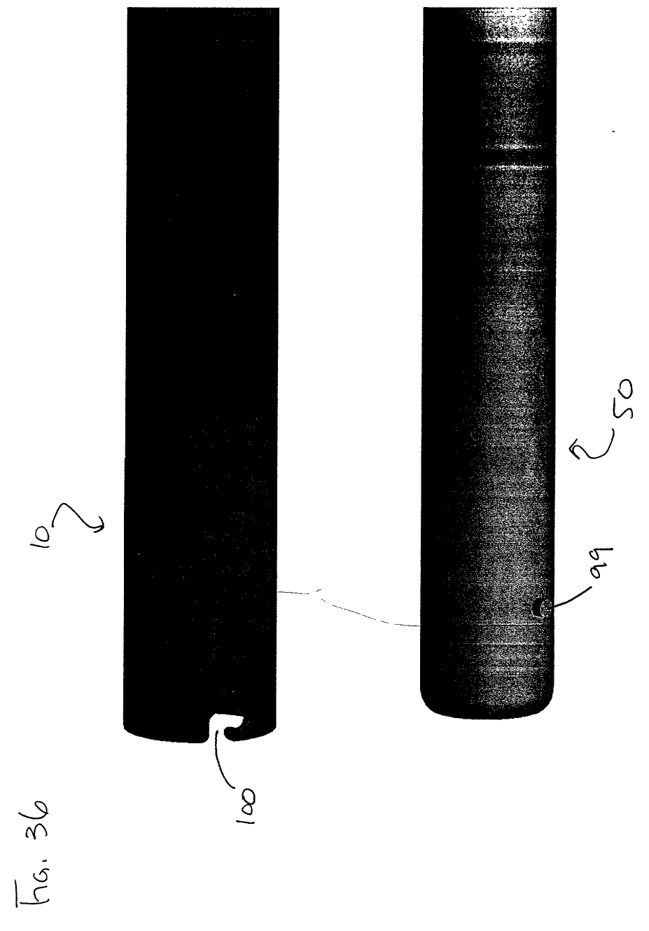

In one embodiment, the guide 10 comprises a sealing element 28 (see, e.g., FIG. 33). In general, the sealing element 28 is positioned within the guide lumen so as to prevent bodily fluids from the target tissue 45 and drug delivered from a drug delivery device 50 positioned within the guide 10 from flowing back into the guide 10. The sealing element 28 can be manufactured from any suitable material that is substantially non-reactive with bodily fluids or tissue and substantially non-reactive with the drug formulation to be delivered using the system of the invention. For example, the sealing element material can be a soft, resilient, self-lubricating elastomeric material, such as silicone rubber. The sealing element can be provided as a separate element that is attached to the guide inner wall, or may be a continuous extension of the material of the guide inner wall. In addition to providing a liquid-proof seal, one sealing element or a plurality of sealing elements can also serve to stably position a drug delivery device within the guide.

In one embodiment, the sealing element is a ring-like structure, where the outer diameter of the sealing element is associated with the inner wall of the guide. The sealing element defines a central passage through which the drug delivery device is removably inserted. The central passage is preferably of a size sufficiently large to accommodate insertion of the drug delivery device without tearing or otherwise damaging the sealing element or damaging the drug delivery device, but sufficiently small so that, following insertion of the drug delivery device, a substantially liquid-tight seal is formed between the sealing element inner surface and the portion of the drug delivery device with which the sealing element communicates. The sealing element may contain or be coated with materials to facilitate smooth insertion and removal of the drug delivery device.

The sealing element (e.g., the inner surface of the sealing element passage) can be shaped to facilitate insertion of the drug delivery device and/or to accommodate the shape of the drug delivery device portion with which it communicates. For example, the sealing element inner surface can be beveled to receive a portion of the drug delivery device, so that the drug delivery device is seated within the sealing element inner surface wall. In another example, the body of the sealing element can taper in thickness toward the central passage, e.g., the sealing element body is thicker where it communicates with the guide and is relatively thinner at the edge of the central passage. In this latter embodiment, the tapered sealing element can be designed to flex upon insertion of the drug delivery device, so that a portion of a side wall of the sealing element contacts a portion of the drug delivery device, providing an increased area of contact between the sealing element and the drug delivery device.

As exemplified in FIG. 33, the guide 10 can comprise a plurality of sealing elements 28, and can be positioned at various points within the guide lumen. In one embodiment, the guide comprises at least one sealing element positioned within a distal portion of the guide lumen, e.g., so as to provide a liquid-proof seal with a drug delivery catheter 60 positioned within the guide lumen. The sealing element can be positioned, for example, at or near the extreme distal end of the guide. The size of the outer diameter and dimensions of the central passage are varied according to the dimensions of the guide 10 and drug delivery device 50.

The sealing element design can also be varied according to the implantation methods used and the treatment site to be accessed. For example, the guide can be implanted with the drug delivery device positioned within the guide, so that the sealing elements provides a liquid-proof seal during implantation. Alternatively, the guide can be implanted prior to insertion of the drug delivery device. In this latter method, it may be desirable to provide the guide with one or more sealing elements that inhibit flow of bodily fluids into the guide during implantation. For example, the guide can comprise a sealing element positioned within a distal portion of the guide lumen, where the sealing element is designed to substantially inhibit flow of bodily fluids or other liquids into the guide. Exemplary sealing elements that can facilitate inhibition of liquid entry into the guide lumen include sealing elements that define a relatively small central passage. In this embodiment, the sealing element is designed of a flexible material or is tapered in thickness toward the central passage to allow for insertion of a drug delivery device. Alternatively, the self-sealing element may comprise a central passage that is not simply empty space, but rather comprises a self-sealing material, e.g., the self-sealing material is positioned within at least a central portion of the sealing element. The self-sealing material positioned within the sealing element central passage is such that a drug delivery device can be readily inserted through the sealing element central passage and, upon withdrawal of the drug delivery device, re-seals to substantially inhibit flow of liquid into the guide lumen. The distal end of the drug delivery device (e.g., the distal end of the drug delivery catheter) can be fashioned for use with such self-sealing sealing elements (e.g., by providing the drug delivery catheter with a tapered or sharpened distal end).

Drug Delivery Device

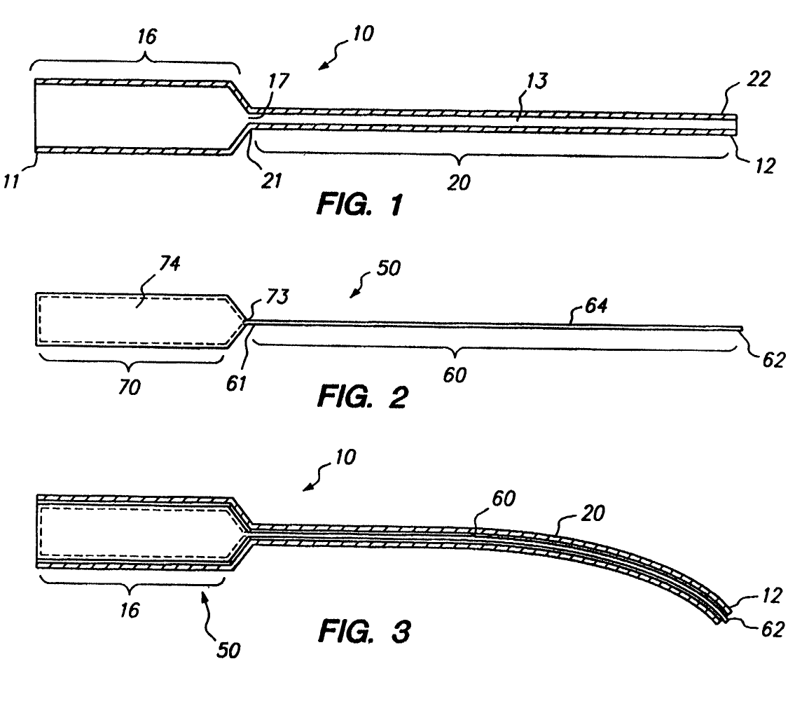

The drug delivery device 50 minimally comprises a drug release device 70 and, in a preferred embodiment, further comprises and a drug delivery catheter 60 (see, e.g., FIGS. 2 and 28). The proximal end 61 of the drug delivery catheter 60 is attached to the drug release device 70 so that the lumen of the drug delivery catheter 60 is in communication with an orifice 73 such that drug contained in the reservoir 74 can move through orifice 73 and into the drug delivery catheter 60 and out the tip of the drug delivery catheter distal end 62.

In an alternative embodiment, the drug delivery device comprises a leash that facilitates retrievable positioning of the drug delivery device at any site within the lumen of the guide. In this latter embodiment, the drug delivery device may further comprise a drug delivery catheter.

Each of the components of the drug delivery device will now be described in more detail.

Drug Release Device

The drug delivery device of the invention can be designed for use in conjunction with any of a variety of drug release devices. In general, the drug release devices suitable for use in the invention comprise a reservoir 74, which reservoir retains a drug formulation therein. The drug release device can be selected form any of a variety of conventional drug release devices that are conventionally used as an external element (e.g., an external pump) or implanted element of a drug delivery system. In a preferred embodiment, the drug release device is a controlled drug release device. Controlled drug release devices suitable for use in the present invention generally can provide for delivery of the drug from the reservoir 74 at a selected or otherwise patterned amount and/or rate through a drug delivery catheter 60 and to a treatment site in the subject.

Release of drug from the reservoir, particularly controlled release of drug from the reservoir, can be accomplished in any of a variety of ways according to methods well known in the art, e.g., by incorporation of drug into a polymer that provides for substantially controlled diffusion of drug from within the polymer, incorporation of drug in a biodegradable polymer, providing for delivery of drug from an osmotically-driven device, etc. Drug can be delivered through the drug delivery catheter to the treatment site as a result of capillary action, as a result of pressure generated from the drug release device, by diffusion, by electrodiffusion or by electroosmosis through the device and/or the catheter.

The reservoir 74 of the drug release device 70 is preferably made of an impermeable material that is sufficiently strong to ensure that it will not leak, crack, break or distort so as to expel its active agent contents under stresses it would be subjected to during use, e.g., due to physical forces exerted upon the drug release device as a result of movement by the subject or physical forces associated with pressure generated within the reservoir associated with drug delivery through the drug delivery catheter. Reservoir 74 must also be chemically inert (e.g., does not react with the active agent formulation) and is preferably biocompatible (e.g., where the device is implanted, it is substantially non-reactive with respect to a subject's body or body fluids).

Suitable materials for reservoir 74 generally comprise a non-reactive polymer or a biocompatible metal or alloy. Suitable polymers include, but are not necessarily limited to, acrylonitrile polymers such as acrylonitrile-butadiene-styrene polymer, and the like; halogenated polymers such as polytetrafluoroethylene, polychlorotrifluoroethylene, copolymer tetrafluoroethylene and hexafluoropropylene; polyimide; polysulfone; polycarbonate; polyethylene; polypropylene; polyvinylchloride-acrylic copolymer; polycarbonate-acrylonitrile-butadiene-styrene; polystyrene; and the like. Further exemplary polymers are described in The Handbook of Common Polymers, Scott and Roff, CRC Press, Cleveland Rubber Co., Cleveland, Ohio. Metallic materials suitable for use in the reservoir 74 of the drug release device 70 include stainless steel, titanium, platinum, tantalum, gold and their alloys; gold-plated ferrous alloys; platinum-plated titanium, stainless steel, tantalum, gold and their alloys as well as other ferrous alloys; cobalt-chromium alloys; and titanium nitride-coated stainless steel, titanium, platinum, tantalum, gold, and their alloys.

A reservoir made from titanium or a titanium alloy having greater than 60%, often greater than 85 % titanium is particularly preferred for the most size-critical applications, for high payload capability and for long duration applications and for those applications where the formulation is sensitive to body chemistry at the implantation site or where the body is sensitive to the formulation. Preferred reservoir materials maintain at least 70% active agent after 14 months at 37° C. and have a shelf stability of at least about 9 months, or more preferably at least about two years, at about 2° C. to 8° C. Most preferably, the drug delivery devices are designed for storage with drug at room temperature. Where unstable formulations are in reservoir 74, e.g., protein and/or peptide formulations, the metallic components to which the formulation is exposed are preferably formed of titanium or its alloys as described above.

Drug release devices suitable for use in the drug delivery devices of the invention may be based on any of a variety of drug delivery systems. For example, the drug release device can be based upon a drug diffusion system, e.g., where the drug is incorporated into a polymer, and the polymer is provided within a drug-impermeable reservoir 74 that is communication with a drug delivery catheter 70. In one embodiment, the polymer provides for release of drug concomitant with degradation of a drug-impregnated polymeric material (e.g., a biodegradable, drug-impregnated polymeric material). In other embodiments, the drug release device is accomplished by osmotic pumps, electrodiffusion, electroosmosis, vapor pressure pumps, electrolytic pumps, effervescent pumps, piezoelectric pumps, erosion-based systems, diffusive systems, etc.

Controlled release of drug can be accomplished by the design of the drug formulation present in the drug delivery device (e.g., within the drug delivery device reservoir or within the drug delivery catheter), the design of the drug release device, and/or the design of the drug delivery catheter. For example, the catheter can be loaded with polymer that provides for controlled diffusion of drug from the drug reservoir.

Drug release devices based upon a mechanical or electromechanical infusion pump, are also suitable for use with the present invention. Examples of such devices include those described in, for example, U.S. Pat. Nos. 4,692,147; 4,360,019; 4,487,603; 4,360,019; 4,725,852, and the like. In general, the present invention can be used in conjunction with refillable, non-exchangeable pump systems. In this latter context the present invention provides several advantages, including improved and repeated access to a treatment site, as well as the elimination of fluid coupling issues normally associated with the conventional use of such devices.

In one embodiment, the drug release device is a controlled drug release device in the form of an osmotically-driven device. Preferred osmotically-driven drug release systems are those that provide for release of drug at a rate of about 0.01 μg/day to about 100 mg/day, which drug can be delivered at a volume rate of from about 0.01 μl/day to about 100 μl/day, preferably about 0.04 μl/day to about 10 μl/day, generally about 0.2 μl/day to about 2.0 μl/day. Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693, 5,728,396; and the like.

In one embodiment the controlled drug release device is an osmotic pump, e.g., an osmotic pump similar to that described in U.S. Pat. No. 5,728,396. In one embodiment of particular interest, the osmotic pump is a DUROS™ osmotic pump. In general, osmotic pumps operate by imbibing fluid from the outside environment and releasing corresponding amounts of the therapeutic agent. The reservoirs of osmotic pumps can be a single chamber, or can be divided into two chambers (e.g., a piston can separate the two chambers). Where the pump comprises two chambers, the first chamber (which lies within one portion of the drug release device reservoir) contains a fluid-imbibing agent, and the second chamber (which lies within a second portion of the drug release device reservoir) contains a therapeutic agent. The fluid-imbibing agent in the first chamber is isolated from the active agent in the second chamber. Where a piston serves to separate the two chambers, the piston is capable of sealably moving under pressure within the reservoir.

A back-diffusion regulating outlet defines an end of the drug-containing second chamber of the osmotic pump. An exemplary back-diffusion regulating outlet is one based on a male threaded member in a mating relationship with the smooth interior surface of the reservoir wall defining the sidewalls of the first chamber, which threaded member forms a helical flow path between the mating surfaces of the back-diffusion regulating outlet and the reservoir through which therapeutic agent from the second chamber can flow. The pitch, the amplitude, and the cross-sectional area and shape of the helical path formed are factors that affect both the efficiency of path preventing back-diffusion of external fluid into the second chamber and the back pressure in the device. The geometry of outlet also prevents water diffusion into the reservoir. In general, the characteristics of the flow path are selected so that the length of the helical flow path and the velocity of flow of active agent therethrough is sufficient to prevent back-diffusion of external fluid through the flow path without significantly increasing the back pressure, so that the release rate of the active agent is primarily governed by the osmotic pumping rate. Alternatively or in addition, where the drug delivery device comprises a drug delivery catheter, the drug delivery catheter can be designed to serve as a back diffusion regulating element.

The first chamber comprises a water-swellable semipermeable membrane. The material of the semipermeable membrane is selected so that it is capable of imbibing between about 0.1% and 200% by weight of water. The semipermeable membrane imbibes fluid to generate a force transferable to the drug-containing second chamber of the pump, thus forcing drug within the second chamber out of the orifice of the second chamber at a controlled rate. The polymeric materials from which the semipermeable membrane may be made vary based on the pumping rates and a device configuration requirements and include but are not limited to plasticized cellulosic materials, enhanced polymethylmethacrylate such as hydroxyethylmethacrylate (HEMA) and elastomeric materials such as polyurethanes and polyamides, polyether-polyamide copolymers, thermoplastic copolyesters and the like.

Drug Delivery Catheter

The drug delivery catheter 60 is generally an hollow tube having a proximal end 61 associated with the drug release device 70 and a distal end 62 for delivery of drug to a desired treatment site. The drug delivery catheter 60 can be provided as an extended orifice from the drug release device 70, e.g., the catheter 60 can be extruded from the body of the drug release device 70 itself so that the catheter is an extension of the material of the wall of the drug release device. Alternatively, the drug delivery catheter can be provided as a component separate from the body of the drug release device 72, which is attachable to the drug release device to, for example, provide for flow of drug through orifice 73 and into the catheter 60. In this latter embodiment, it may be desirable to include a component that facilitates attachment of the drug delivery catheter to the drug release device and/or stabilize such attachment, e.g., substantially diminish movement of the drug delivery catheter in a direction perpendicular to the longitudinal axis of the drug release device (e.g., to provide strain relief), so as to reduce risk of breakage of the drug delivery catheter at the attachment site.

The drug delivery catheter 60 comprises a lumen having a diameter that can be equal to, or can be greater or less than, the diameter of the drug release device orifice 73. Where the drug release system used in the drug delivery device dispenses drug by convection (as in, e.g., osmotic drug delivery systems), the orifice size as well as the size of the lumen of the drug delivery catheter leading from the reservoir of the drug release system can be designed as described by Theeuwes (1975) J. Pharm. Sci. 64:1987-91. The orifice design criteria define the characteristics of the back-diffusion regulating element.

The drug delivery catheter 60 can have substantially the same inner and outer diameters throughout its length, or the inner diameter and/or outer diameter can vary along the catheter's length. Likewise, the walls of the drug delivery catheter can be of substantially the same thickness throughout its length, or can vary in thickness throughout the catheter's length. For example, the catheter can have an inner diameter that is equal to or greater than the diameter of the orifice at its proximal end, with a constriction smaller than the orifice of the release device at its distal end such that at least the inner diameter of the catheter tapers to a smaller drug delivery outlet at the distal end.

The drug delivery catheter 60 can comprise a catheter body 64 having any of a variety of dimensions and geometries, which are selected to be most suitable for the intended use of the drug delivery device (e.g., the desired treatment site, the amount of drug to be delivered, the drug release device to be used in conjunction with the drug delivery catheter, the desired means of attachment of the catheter to the drug release device to facilitate flow of drug from the drug release device to the catheter, etc.). The catheter body 64 will typically have a length in the range from about 1 cm to 150 cm, usually having a length in the range from about 2-5 cm up to about 50 cm. The outside diameter of the catheter body will typically be in the range from about 0.1 mm (0.3 F) to 2 mm (6 F), usually being in the range from about 0.125 mm (0.4 F) to about 1 mm (3 F). In one embodiment, the drug delivery catheter has an outer diameter of about 0.009″. The drug delivery catheter body will define an inner lumen typically having a diameter in the range from about 0.025 mm to 1.5 mm, usually being in the range from about 0.05 mm to 1 mm, with catheters having larger outside diameters usually having larger lumen diameters. In one embodiment, the drug delivery catheter has an inner diameter of about 0.009″.

In general, the drug delivery catheter body has a length in the range from about 1 cm to about 200 cm, usually from about 15 cm to about 40 cm; an outside diameter in the range from about 0.125 mm (0.4 F) to about 3 mm, usually from about 0.66 mm (2 F) to about 0.5 mm; and an inside diameter in the range from about 0.05 mm to about to about 2 mm, usually from about 0.075 mm to about 0.5 mm (2 F). The outside diameter of the drug delivery catheter is less than the inside diameter of a lumen of the guide body 20 that is to be used in conjunction with the drug delivery device. It should also be noted that the drug delivery orifice 73 may be provided in the drug release device distal end 72 as a distinct opening or as a series of openings, e.g., as in the context of a rate-limiting membrane, which membrane defines a plurality of openings through which drug may flow from the drug reservoir 74. In either embodiment, the inner diameter of at least the proximal end 61 is of a size sufficient to provide a leak-resistant or leak-proof drug flow path from the reservoir 74 through the drug delivery catheter lumen.

The dimensions of the drug delivery device (e.g., dimensions of the drug release device, drug delivery catheter, etc.) can vary according to a variety of factors such as the treatment site for drug delivery, the guide with which the drug delivery device is to be used, the desired drug delivery rate, the length of the course of treatment, etc.

The drug delivery catheter may be produced from any of a variety of suitable, substantially impermeable materials, and may be manufactured from the same or different material as the impermeable reservoir of the drug release device. Impermeable materials suitable for use in production of the controlled drug release device as described above are generally suitable for use in the production of the drug delivery catheter. The drug delivery catheter can generally be made from a relatively stiff catheter material, since the guide will provide protection of tissue during placement of the drug delivery device, and thus avoid substantial tissue damage and trauma to the patient. Exemplary materials from which the drug delivery catheter can be manufactured include, but are not necessarily limited to, polymers; metals; glasses; polyolefins (high density polyethylene (HDPE), low density polyethylene (LDPE), linear low density polyethylene (LLDPE), polypropylene (PP), and the like); nylons; polyethylene terephtholate; silicones; urethanes; liquid crystal polymers; PEBAX™; HYTREL™; TEFLON™; perflouroethylene (PFE) perflouroalkoxy resins (PFA); poly(methyl methacrylate) (PMMA); multilaminates of polymer, metals, and/or glass; nitinol; and the like. In one embodiment, the drug delivery catheter is manufactured from a nickel titanium alloy (NITINOL™).

The drug delivery catheter can comprise additional materials or agents (e.g., coatings on the external or internal catheter body surface(s)) to facilitate placement of the drug delivery catheter within the guide and/or to provide other desirable characteristics to the catheter. For example, the drug delivery catheter can be coated with silver or otherwise coated or treated with antimicrobial agents, thus further reducing the risk of infection at the treatment site.

In general, the material of the drug delivery catheter is selected so as to provide the catheter with the desired degree of flexibility or stiffness. The flexible or stiff nature of the drug delivery catheter can be substantially the same throughout its length, or can vary over its length, e.g., a distal portion of the catheter may be more flexible or more stiff relative to the proximal portion. In general, the drug delivery catheter body 64 is sufficiently flexible so that it can pass through any tortuous bends in the implanted guide 10, so as to facilitate movement of the catheter through the twists and turns that may be present in the access pathway to the treatment site. The drug delivery catheter body is preferably sufficiently stiff so as to allow for pushing of the catheter through the guide, particularly for pushing the drug delivery catheter through such tortuous bends in the guide. Alternatively or in addition, a support member (e.g., a guide wire) may be provided, e.g., around the outside of the catheter body, to facilitate pushing of the catheter through the guide. The use of such a support member can allow for use of less stiff materials for the drug delivery catheter body.

The distal end of the drug delivery catheter can be shaped so as to allow for smooth passage through the guide, particularly where the guide is in a tortuous bending configuration. For example, the distal end of the catheter can be provided as a rounded tip that allows for the catheter to move smoothly around a guide bend (e.g., where a square-ended catheter tip might catch on the sidewalls of the guide, thus frustrating positioning of the drug delivery device).

A number of variations on this basic drug delivery catheter design are contemplated by the present invention. For example, the distal end of the drug delivery catheter may optionally end in a one-way valve such as a duck bill valve to prevent retrograde flow in the drug delivery catheter, with external pressure at that distal end. Alternatively or in addition, the distal end may comprise a porous plug that serves as a filter element preventing particulate matter (including bacteria) from exiting from the drug delivery catheter and into the treatment site. The drug delivery catheter can also be provided as a multi-lumen catheter, where at least one lumen serves as a drug delivery conduit. In the multi-lumen embodiment, one of the lumen can define a space through which a guide wire is threaded to facilitate positioning of the drug delivery device within a lumen of a guide. The drug delivery catheter may comprise a single drug outlet at the distal end for delivery of drug at or near a treatment site, or may comprise a plurality of such drug outlets (e.g., in the form of side holes along a portion of the distal end of the catheter).

In use, the drug delivery catheter 60 is threaded into the guide so that the distal end 62 of the drug delivery catheter defining a drug delivery outlet is positioned for delivery of drug at a treatment site. In one embodiment, the drug delivery catheter 60 is primed with drug, e.g., is substantially pre-filled with drug. Priming of the drug delivery catheter reduces delivery start-up time, i.e., time related to movement of the drug from the drug release device to the distal end of the drug delivery catheter. This feature is particularly advantageous where the drug release device of the drug delivery device releases drug at relatively low flow rates (e.g., 0.4 μl/day). The drug used to prime the drug delivery catheter may be the same drug that is delivered from the drug release device of the drug delivery device, or may be a different drug or different formulation of the drug, e.g., the drug delivery catheter itself may provide for a component of the therapeutic regimen.

Sealing Element

In one embodiment, the drug delivery device 50 comprises a sealing element 28 (see,. e.g., FIG. 28). In general, the sealing element 28 is positioned on the outer surface of the drug delivery device so, when positioned within a guide, backflow of bodily fluids from the target tissue 45 and/or drug delivered from the drug delivery device 50 into the guide lumen is substantially inhibited. The materials suitable for manufacture of the sealing element of the drug delivery device are substantially the same as those suitable for manufacture of sealing elements used within a guide as described above.

The sealing element can be provided as a separate element that is attached to the drug delivery device outer wall (e.g., an O-ring positioned around the outer wall of the drug delivery device), or may be a continuous extension of the material of the drug delivery device outer wall. The drug delivery device can comprise a single sealing element or a plurality of sealing elements, and such sealing element(s) can be positioned along any portion of the drug delivery device. In one embodiment, at least one sealing element is positioned at a distal portion of the drug deivery device, e.g., at or near the distal end of a drug delivery catheter of the drug delivery device. In addition to providing a liquid-proof seal, the sealing element(s) can also serve to stably position the drug delivery device within the guide.

Leash Embodiment

In one embodiment, the drug delivery device comprises a leash for retrieving the drug delivery device. In general, the leash comprises a proximal end and a distal end, where the distal end is attached to a portion of the drug delivery device 50. The proximal end of the leash is retained at the implantation site or access site in the subject, and may be retained within a portion of the distal end of the guide. Drug delivery devices comprising such a leash can be positioned at any point within the lumen of the guide (e.g., at a site any distance from an access or implantation site at which the drug delivery device is initially introduced into the guide). In this latter embodiment, the drug delivery device can further optionally comprise a drug delivery catheter.

The leash can be made from any suitable material that is of sufficient strength to allow retrieval of the drug delivery device from within the guide lumen. Exemplary materials include multifilament strands (e.g., nylon), metals (e.g., stainless steel, nickel titanium, beryllium, copper, nickel, and alloys thereof), polymers, glasses, plastics, and other suitable materials, which typically can be selected from the same or similar materials described above for manufacture of the catheter. In one embodiment, the leash is sufficiently stiff to allow pushing and position of the drug delivery device at a selected position along the guide. The position of the drug delivery device along the guide may affect drug delivery rate. For example, in the case of a diffusional drug delivery system, a drug delivery pathway is defined by the distance from the drug delivery device to the treatment site. By selecting the drug delivery pathway length, the drug delivery rate can be modified according to Equation I above, where the length of the drug delivery pathway is L, and the guide inner diameter is A.

Drugs for Delivery Using the Drug Delivery Device

Any of a wide variety of drugs can be delivered using the drug delivery system of the invention. Drugs suitable for delivery are generally provided as flowable formulations, and are generally provided as liquids or semisolids. The drugs may be anhydrous or aqueous solutions, suspensions or complexes, and may be formulated with pharmaceutically acceptable vehicles or carriers, as well as additional inert or active ingredients. The drugs of formulations suitable for delivery using the invention may be in various forms, such as uncharged molecules, components of molecular complexes or pharmacologically acceptable salts. Also, simple derivatives of the agents (such as prodrugs, ethers, esters, amides, etc.) that are easily hydrolyzed by body pH, enzymes, etc., can be employed. Preferably the agents are formulated so as to remain stable for long periods of storage on the shelf or under refrigeration, as well as for long periods stored in an implanted drug delivery system of the invention.

Of particular interest is the treatment of diseases or conditions that require long-term therapy, e.g., chronic and/or persistent diseases or conditions for which therapy involves treatment over a period of several days (e.g., about 3 days to 10 days), to several weeks (e.g., about 3 or 4 weeks to 6 weeks), to several months or years, up to including the remaining lifetime of the subject. Subjects who are not presently suffering from a disease or condition, but who are susceptible to such may also benefit from prophylactic therapies using the devices and methods of the invention.

Use of Guide and Drug Delivery Device

The drug delivery device and guide of the invention can be used in a wide variety of subjects, including humans. The guide and delivery device can be implanted at any convenient site within the subject's body and oriented for delivery to any desired treatment site. Generally, at least a portion of the proximal end of the guide is retained at an accessible, subcutaneous site, (e.g., under the skin of the arm, shoulder, neck, back, or leg), or at a subcutaneous site within a body cavity (e.g., within the mouth). The proximal end of the guide can be at a site close (e.g., within a few centimeters, e.g., within about 2 cm), or at a site relatively distant (e.g., more than about 30 cm, generally greater than about 50 cm to 100 cm) from the treatment site, and thus from the ultimate site of drug delivery. A single guide and/or drug delivery device, or two or more guides and/or drug delivery devices can be implanted in a subject during the course of a therapeutic program.

The guide is generally designed to remain implanted in the subject for an extended period, e.g., from several days, to several weeks, months, or years, and can be designed to be substantially permanently implanted in the subject (e.g., for the subject's remaining lifespan). The drug delivery devices are generally designed to remain partially or substantially completely implanted, preferably substantially completely implanted, within the guide for a predetermined administration period, and are normally removed and replaced at the end of such administration period. However, the drug delivery devices can be designed to remain implanted within the guide for extended periods.

The devices of the present invention are preferably rendered sterile prior to use. This may be accomplished by separately sterilizing each component, e.g., by gamma radiation, steam sterilization or sterile filtration, etc., then aseptically assembling the final system. Alternatively, the devices may be assembled, then terminally sterilized using any appropriate method.

Implantation of the Guide

Insertion of the guide and drug delivery device can be accomplished using methods and tools that are well known in the art. Insertion of the guide is accomplished in a manner similar to insertion of any of a variety of catheters, e.g., under aseptic conditions with at least some local or general anesthesia administered to the subject. Where the guide comprises radiopaque material, insertion of the guide and/or guide body can be monitored by X-ray or other means of visualization of the guide insertion process. The guide and delivery device can be positioned for drug delivery in the subject in separate steps, or in a single step as a complete drug delivery system. The guide and/or drug delivery device can optionally comprise one or more anchoring elements, e.g., rings or ears (see, e.g., FIG. 4), for retaining the guide and/or drug delivery device at a local site.

Guide and Drug Delivery Device Combinations to Provide a Drug Delivery System

The present invention encompasses any of a variety of combinations of guides and drug delivery devices. The combination of the guide and drug delivery device can be varied according to a variety of factors such as the specific treatment site to which drug is to be delivered, the drug formulation to be delivered, etc. The ability to vary the characteristics of the guide material and the drug delivery device material, particularly the material of a drug delivery catheter used in connection with the drug delivery device, provides the clinician or other health professional with a wide variety of drug delivery systems that can be selected according to the needs of the patient.

In general, the system of the invention comprises a drug delivery device, wherein all or at least a portion of the drug delivery device is positioned within a guide so that a drug delivery pathway is defined from a reservoir of drug within the drug delivery device to the treatment site. In one embodiment of interest, the drug delivery device comprises a drug delivery catheter. In use, the catheter of the drug delivery device is inserted into the guide lumen, and all or at least a portion of the drug delivery device is stably positioned within the guide. In this embodiment, it is important that the drug delivery catheter and the guide are manufactured from, or comprise coatings of, materials that facilitate sliding of the outer wall of the drug delivery catheter within the lumen defined by the inner wall of the guide. For example, the guide inner wall and/or outer diameter of the drug delivery device (e.g., the outer wall of a drug delivery catheter) comprises a fluorenated polymer (e.g., teflon), an olefin (e.g., HDPE), a silicon-based coating, a hydrophilic coating, PARYLENE™, etc.

Exemplary embodiments of such variations, and exemplary methods for their implantation, are described in more detail below.

Relatively Flexible Guide with a Relatively Stiff Drug Delivery Catheter

In one embodiment, the guide comprises a relatively soft or flexible guide body. In one access system of interest, the relatively flexible guide is used with a drug delivery device having a drug delivery catheter comprising relatively stiffer materials. The relatively soft or flexible guide body in this embodiment is sufficiently flexible so that it is well-tolerated within the body, is not prone to breakage or leakage, and provides a protective function to the surrounding tissue during insertion of the relatively stiff drug delivery catheter.

Implanting Relatively Flexible Guide

Implantation of a relatively soft guide can be accomplished according to any of a variety of strategies. For example, the access pathway may be defined using a tunneling device 85, such as a rigid or semi-rigid cannula or trocar (see, e.g., FIGS. 29 and 30). The tunneling device 85 can be substantially straight throughout its length (as exemplified in FIG. 29, or such as a splittable needle), or may be shaped to provide for positioning of the guide so as to define a non-linear pathway (as exemplified in FIG. 30). The tunneling device can be used to bore through tissue to access a site of delivery (e.g., an intrathecal space within the spine) so that the distal end of the tunneling device is positioned adjacent or within the desired treatment site. A proximal portion of the tunneling device is retained at a readily accessible site, e.g., an external or subcutaneous site. The lumen of the tunneling device defines a conduit from the accessible site to the treatment site.

The flexible guide is positioned within the cannula lumen either during initial insertion of the cannula, or in a subsequent step in which the flexible guide is threaded through the cannula. Where the guide is inserted into the tunneling device in a subsequent step, it may be desirable to deliver the guide through the tunneling device lumen using a wire, particularly where the guide is so flexible that the material of the guide body cannot be readily pushed through the tunneling device lumen. For example, a wire or stylet can be positioned within the guide lumen, and the wire and guide inserted into the tunneling device lumen as exemplified in FIG. 30.

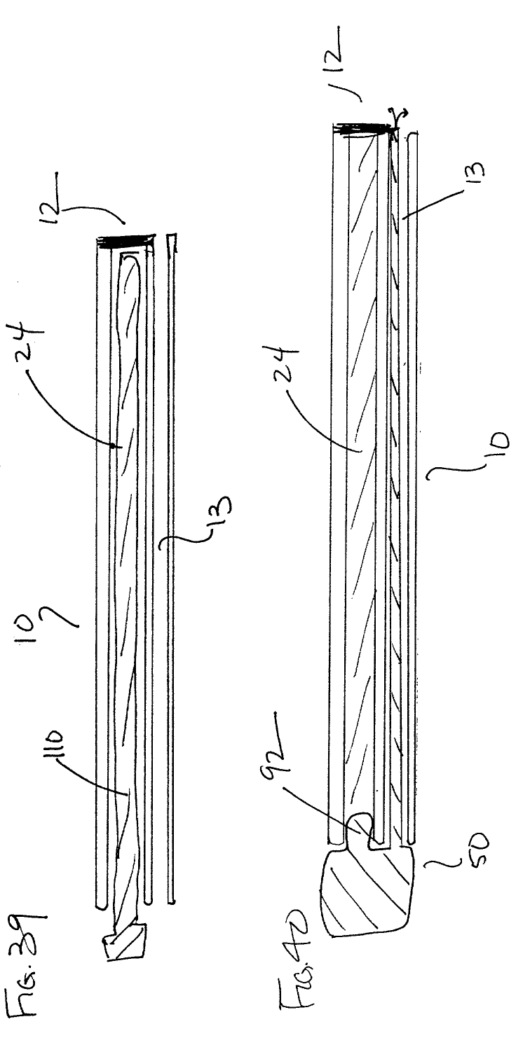

In one embodiment, the guide is designed to facilitate easy withdrawal of the insertion cannula following implantation. For example, where the guide to be used comprises a guide chamber or other element positioned at the guide proximal end, the guide chamber or other element is provided as an attachable element. As exemplified in FIGS. 37 and 38, where the guide 10 comprises a guide chamber 16 at the proximal end, the guide chamber 16 is attached to the guide proximal end by means of an attaching element 30. The attaching element 30 can be any suitable element for facilitating permanent or reversible connection between the guide body and the guide chamber. Exemplary attaching elements include, but are not necessarily limited to, a press-fit lock, a threaded element, a bayonet connector, luer lock, snap fit tab and recess, etc. In this embodiment, the guide 10 without the attached guide chamber 16 can be positioned within the lumen of cannula 85 during initial insertion of the cannula, or in a subsequent step in which the guide is threaded through the cannula. Once the guide is in place, the cannula can be withdrawn over the body of the guide 10, and the guide chamber 16 attached by means of the attaching element 30.

Alternatively implantation of a relatively flexible guide can be accomplished using a wire, stylet, or other reinforcing element that imparts substantial stiffness to the guide for insertion to the treatment site. The reinforcing element can be introduced into the guide lumen, and the guide and reinforcing element combination implanted into the subject to place the guide distal end at the treatment site. The reinforcing element can be then be withdrawn, leaving the guide in place.

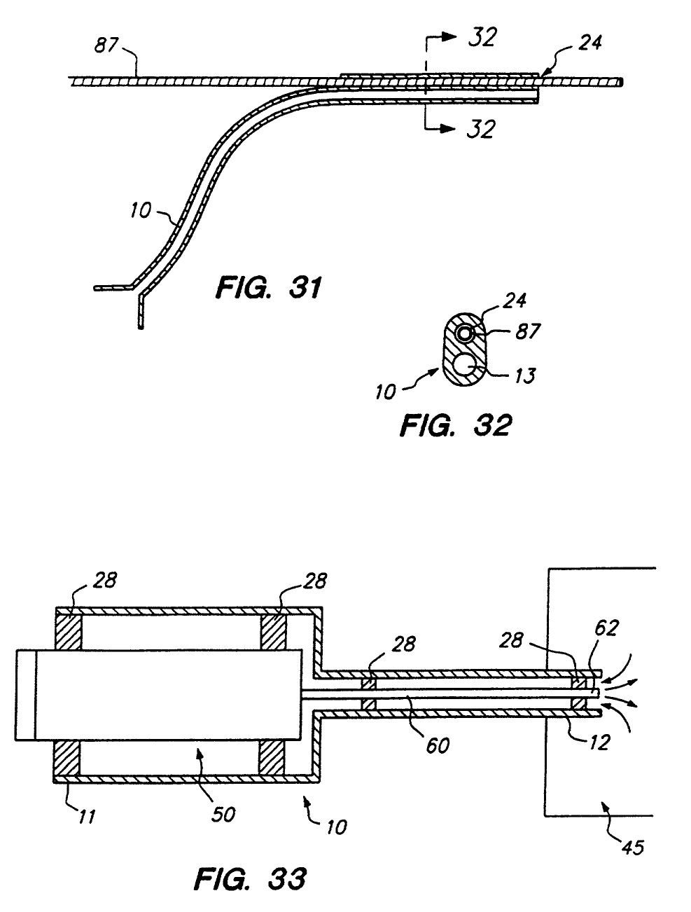

The guide can be readily adapted for use with a reinforcing element. For example, the guide can comprise a reinforcing element channel 24 (see, e.g., FIGS. 31 and 32). The reinforcing element channel 24 can be positioned adjacent all or a portion of the guide body. In the embodiment illustrated in FIGS. 31 and 32, the guide and wire are provided as a monorail type system, where the guide rides over the reinforcing element (exemplified by wire 87).

In another embodiment, the reinforcing element channel 24 is adapted for use with a pushing element, such as a mandrel, to facilitate implantation. As exemplified in FIGS. 39 and 40, the reinforcing element channel 24 is closed at the guide distal end 12. During implantation a mandrel 110 or other pushing element is inserted into the reinforcing element channel 24 to facilitate positioning of the guide distal end at a treatment site. Once the guide is in place, the mandrel 110 is removed. The empty reinforcing element channel 24 can then be filled, with a liquid, semi-solid, or solid material, which material preferably comprises an antimicrobial agent (e.g., a bacteriostatic and/or bactericidal agent). In addition, or alternatively, the inner wall of the reinforcing element channel is coated with antimicrobial coating. The drug delivery device 50 can be positioned within the guide lumen 13 during implantation, or inserted into the guide lumen 13 following implantation (e.g., before or after withdrawal of the mandrel). In one embodiment, the drug delivery device is stably positioned within the guide through a stable positioning element (e.g., a locking/docking mechanism) that utilizes a portion of the reinforcing element channel 24.

The guide can be relatively flexible throughout its length, or may be relatively flexible for only a portion of its length, e.g., relatively flexible over a distal portion of the guide body. Where the guide is flexible only over a portion of its length and comprises a reinforcement channel, the reinforcement element channel can be positioned adjacent only the relatively flexible portion of the guide.

Positioning of Relatively Stiff Catheter in Relatively Flexible Guide

Drug delivery catheters that are relatively stiff can be readily pushed through the relatively flexible guide, providing for ease in insertion of the catheter into the guide and placement at the treatment site. The use of the relatively flexible guide with a relatively stiff drug delivery catheter is advantageous in that the guide serves to protect the surrounding tissue from the drug delivery catheter. In this embodiment it may be desirable for the distal end of the drug delivery catheter to be blunt, rounded, or tapered to avoid catching of the drug delivery catheter end against an inner wall of the guide and/or to prevent damage to the tissue at or surrounding the treatment site.

Relatively Stiff Guide with Relatively Flexible Drug Delivery Catheter

Alternatively, it may be desirable to use a guide having a relatively stiff guide body with a drug delivery device having a relatively soft or flexible drug delivery catheter. If a guide body is of sufficient stiffness, the guide can be implanted within the subject without the use of, for example, a tunneling device or reinforcement element. Where the guide is relatively stiff, the drug delivery catheter can be relatively more flexible throughout its length or, for example, at a distal portion of the catheter. The flexible drug delivery catheter may be of particular use where it is desirable to deliver drug from the catheter at a point distally beyond the distal end of the guide.

Placement of Drug Delivery Device

Once the guide is in place, then the drug delivery device is positioned within the guide to facilitate delivery of drug from the drug delivery device and to the treatment site. In general, the drug delivery device is placed in the guide by inserting the drug delivery device distal end into the guide lumen to position the drug delivery device for delivery of drug from the device's drug reservoir to the treatment site.

Where the drug delivery device comprises a drug delivery catheter, the catheter can be inserted into the guide lumen and up to and/or through the guide distal end. The drug release device is then positioned at or within the guide proximal end, and may be retained thereat or therein via any of a variety of stable positioning elements as described above. The guide proximal end and the drug release device retained therein are generally retained at a subcutaneous site as described above.

Re-access of the Treatment Site

The guide provides for ready access and re-access to the treatment site, thus providing a conduit for drug delivery, sampling, etc. For example, where the guide is used in conjunction with a drug delivery device, the drug delivery device can be readily positioned within the guide to facilitate delivery of the drug to the. treatment site. When desirable, the drug delivery device can be easily removed, and, where desirable, replaced with a new drug delivery device.

Removal and/or replacement of the drug delivery device can be accomplished using tools and methods that are readily available. For example, where a proximal portion of the drug delivery device and/or guide is retained at a subcutaneous site, the drug delivery device can be removed by first locating the guide proximal end (and/or drug release device proximal end) by fingertip palpation of the subcutaneous site of insertion. After anesthetizing the subject at least locally, an incision is made through the skin and any fibrous capsule tissue surrounding the area of implantation. The end of the device opposite the incision is pushed so that the proximal end of the guide is urged out of the incision. The drug delivery device can then be released from the guide and withdrawn. A replacement drug delivery device, which device may comprise the same or different drug and drug formulation, can then be inserted into the guide as described above. Upon placement of the drug delivery device and securing of the device in the guide, the guide is then urged back into the original incision, and the incision closed. This procedure can be designed so that removal and replacement of drug delivery devices can be performed on an outpatient basis, and with minimal discomfort to the subject.

### EXAMPLES

The following examples are put forth so as to provide those of ordinary skill in the art with a complete disclosure and description of how to make and use various constructs and perform the various methods of the present invention and are not intended to limit the scope of what the inventors regard as their invention nor are they intended to represent or imply that the embodiments described below are all on the only embodiments constructed or tested. Efforts have been made to ensure accuracy with respect to numbers used (e.g., amounts, concentrations, particular components, etc.) But some deviations should be accounted for.

### Example 1

In one embodiment, the guide is a composite of teflon on the inside diameter of the guide and silicone laminated on the outside, and has an outer diameter of about 0.040″ and an inner lumen diameter of about 0.012″. The proximal end of the guide is adapted to receive the distal portion of a drug release portion of a drug delivery device, and includes titanium guide chamber that houses the drug delivery device. The guide is flexible, and is implanted into the subject using a rigid or semi-rigid cannula.

### Example 2

In one embodiment, the drug delivery device is an implantable osmotic pump (e.g., DUROS™) having a drug delivery catheter attached to the a distal portion of the pump so as to provide a drug delivery pathway from the reservoir of the pump and through the catheter. The drug delivery catheter is made from a nickel titanium alloy, and has an inner diameter of about 0.006″, and an outer diameter of about 0.010″.

The invention as shown and described is considered to be the one of the most practical and preferred embodiments. It is recognized, however, that the departures may be made therefrom which are within the scope of the invention and that obvious modifications will occur to one skilled in the art upon reading this disclosure.

What is claimed is:

1. An implantable guide for facilitating repeated access to a treatment site in a subject, the guide comprising:
a proximal end, a distal end, and a guide body, wherein the guide body defines a lumen extending from the guide proximal end to the guide distal end; and
a stable positioning element for stably positioning at least a portion of a drug delivery device within the guide.

2. The implantable guide of claim 1, where at least a portion of the guide body is flexible.

3. The implantable guide of claim 1, wherein the guide comprises an openable valve.

4. The implantable guide of claim 1, wherein the guide comprises a sealing element positioned within the guide lumen.

5. The implantable guide of claim 1, wherein the guide body defines a plurality of lumen.

6. The implantable guide of claim 1, wherein the guide is adapted to accommodate at least two drug delivery devices.

7. The implantable guide of claim 1, wherein the guide further comprises a reinforcing element channel.

8. The implantable guide of claim 1, wherein the guide further comprises an anchoring element.

9. The implantable guide of claim 1, wherein the guide body comprises a material selected from the group consisting of: a polymer, a metal, glass, a polyolefin, nylon, polyethylene terephtholate, silicon, urethane; a liquid crystal polymer and a fluorenated polymer.

10. The implantable guide of claim 1, wherein the guide is shaped for implantation to a treatment to a site that is subcutaneous, intravenous, intrathecal, intraorbital, intraocular, intraaural, intratympanic, intramuscular, intra-arterial, intra-articular, intracavitary, intraductal, intraglandular, intravascular, intranasal, intraperitoneal, intraspinal, epidural, intracranial, intracardial, intrapericardial, peritumoral, or intratumoral.

11. An implantable guide for facilitating repeated access to a treatment site in a subject, the guide comprising:
a proximal end, a distal end, and a guide body, wherein the guide body defines a lumen extending from the guide proximal end to the guide distal end; and
a sealing element for providing a liquid-resistant seal with at least a portion of a drug delivery device positioned within the guide lumen.

12. The implantable guide of claim 11, wherein at least a portion of the guide body is flexible.

13. The implantable guide of claim 11, wherein the guide comprises an openable valve.

14. The implantable guide of claim 11, wherein the guide comprises a stable positioning element for stably positioning at least a portion of a drug delivery device within the guide.

15. The implantable guide of claim 11, wherein the guide body defines a plurality of lumen.

16. The implantable guide of claim 11, wherein the guide is adapted to accommodate at least two drug delivery devices.

17. The implantable guide of claim 11, wherein the guide further comprises a reinforcing element channel.

18. The implantable guide of claim 11, wherein the guide further comprises an anchoring element.

19. The implantable guide of claim 11, wherein the guide body comprises a material selected from the group consisting of: a polymer, a metal, glass, a polyolefin, nylon, polyethylene terephtholate, silicon, urethane; a liquid crystal polymer and a fluorenated polymer.

20. The implantable guide of claim 11, wherein the guide is shaped for implantation to a treatment to a site that is subcutaneous, intravenous, intrathecal, intraorbital, intraocular, intraaural, intratympanic, intramuscular, intra-arterial, intra-articular, intracavitary, intraductal, intraglandular, intravascular, intranasal, intraperitoneal, intraspinal, epidural, intracranial, intracardial, intrapericardial, peritumoral, or intratumoral.

21. A drug delivery device comprising:
a drug release device comprising a reservoir, and an orifice defined by a distal portion of the drug release device;
a substantially hollow drug delivery catheter comprising a drug delivery catheter proximal end and a drug delivery catheter distal end, wherein the drug delivery catheter proximal end is coupled to the drug release device to provide a drug flow pathway from the reservoir, through the orifice, and through a lumen of the drug delivery catheter; and
a stable positioning element for stably positioning at least a portion of the drug delivery device within a guide.

22. The drug delivery device of claim 21, wherein the stable positioning element comprises a recess for receiving a snap fit tab.

23. The drug delivery device of claim 21, wherein the drug delivery device further comprises a sealing element for providing a liquid-resistant seal with a guide.

24. The drug delivery device of claim 21, wherein the drug delivery catheter comprises a material selected from the group consisting of: a polymer, a metal, glass, a polyolefin, nylon; polyethylene terephtholate, silicone, urethane, a liquid crystal polymer, a fluorenated polymer, and nitinol.

25. The drug delivery device of claim 21, wherein the drug delivery catheter comprises nitinol.

26. The drug delivery device of claim 21, wherein the drug delivery catheter is coated with silver or an antimicrobial agent.

27. A drug delivery device comprising:
a drug release device comprising a reservoir, and an orifice defined by a distal portion of the drug release device;
a substantially hollow drug delivery catheter comprising a drug delivery catheter proximal end and a drug delivery catheter distal end, wherein the drug delivery catheter proximal end is coupled to the drug release device to provide a drug flow pathway from the reservoir, through the orifice, and through a lumen of the drug delivery catheter; and
a sealing element for providing a liquid-resistant seal with a guide.

28. The drug delivery device of claim 27, wherein the drug delivery device comprises a stable positioning element for stably positioning at least a portion of the drug delivery device within a guide.

29. The drug delivery device of claim 27, wherein the stable positioning element is a snap fit recess for mating with a snap fit tab.

30. The drug delivery device of claim 27, wherein the drug delivery catheter comprises a material selected from the group consisting of: a polymer, a metal, glass, a polyolefin, nylon, polyethylene terephtholate, silicone, urethane, a liquid crystal polymer, a fluorenated polymer, and nitinol.

31. The drug delivery device of claim 27, wherein the drug delivery catheter comprises nitinol.

32. The drug delivery device of claim 27, wherein the drug delivery catheter is coated with silver or an antimicrobial agent.

33. A system for delivery of drug to a treatment site comprising:
an implantable guide comprising a proximal end, a distal end, and a guide body, wherein the guide body defines a lumen extending from the guide proximal end to the guide distal end;
a drug delivery device, wherein at least a portion of the drug delivery device is removably and stably positioned within the guide lumen; and
a stable positioning element that stably and removably associates the guide with the drug delivery device;
wherein the drug delivery device is positioned for delivery of drug from a drug reservoir of the drug delivery device and through the distal end of the guide lumen to the treatment site.

34. The system of claim 33, wherein the drug delivery device comprises:
a drug release device comprising a drug reservoir and a distal portion defining a drug delivery orifice; and
a drug delivery catheter comprising a drug delivery catheter proximal end and a drug delivery catheter distal end, wherein the drug delivery catheter proximal end is coupled to the drug release device to provide a drug delivery pathway from the drug reservoir, through the orifice, and through a lumen of the drug delivery catheter to the drug delivery catheter distal end.

35. The system of claim 34, wherein the drug release device is selected from the group consisting of a diffusion system, an osmotic pump, an electromechanical pump, an erodible drug-comprising polymer, an electrodiffusion pump, an electroosmotic pump, a piezoelectric pump, a vapor pressure pump, and an electrolytic pump.

36. The system of claim 34, wherein the drug delivery catheter distal end terminates within the guide body.

37. The system of claim 34, wherein the drug delivery catheter distal end terminates at a point distal to the guide distal end.

38. The system of claim 34, wherein the drug delivery catheter is at least partially filled with drug prior to implantation.

39. The system of claim 33, wherein the guide comprises an outer diameter of from about 0.1 mm to 3 mm.

40. The system of claim 34, wherein the drug delivery catheter comprises an inner diameter of from about 0.025 mm to 1.5 mm.

41. The system of claim 34, wherein the drug delivery catheter is stiff relative to the guide body of the guide.

42. The system of claim 33, wherein at least a portion of the guide body is flexible.

43. A system for delivery of drug to a treatment site comprising:
an implantable guide comprising a proximal end, a distal end, and a guide body, wherein the guide body defines a lumen extending from the guide proximal end to the guide distal end;
a drug delivery device, wherein at least a portion of the drug delivery device is removably and stably positioned within the guide lumen; and
a sealing element positioned between an inner wall of the guide lumen and an outer wall of the drug delivery device;
wherein the drug delivery device is positioned for delivery of drug from a drug reservoir of the drug delivery device and through the distal end of the guide lumen.

44. The system of claim 43, wherein the system further comprises a stable positioning element for associating the drug delivery device with the guide.

45. The system of claim 43, wherein the stable positioning element comprises a snap fit tab.

46. The system of claim 43, wherein the stable positioning element comprises a bayonet-style connector.

47. The system of claim 43, wherein the stable positioning element comprises a luer lock.

48. The system of claim 43, wherein the stable positioning element comprises a a threaded coupling member.

49. The system of claim 43, wherein the stable positioning element comprises a press-fit connection.

50. A method for site-specific drug delivery, the method comprising:
implanting in a subject a guide of claim 1, said implanting providing for placement of the guide distal end at a treatment site;
inserting at least a portion of a drug delivery device into the implanted guide, said insertion providing for stable positioning of the drug delivery device at a proximal end of the guide; and
delivering a drug from the drug delivery device to the treatment site from the guide distal end.

51. The method of claim 50, wherein the drug delivery device comprises a drug release device and a drug delivery catheter, and wherein the drug release device is retained at the proximal end of the guide and the drug delivery catheter is positioned within the guide lumen.

52. The method of claim 50, wherein the treatment site is subcutaneous, intravenous, intrathecal, intraorbital, intraocular, intraaural, intratympanic, intramuscular, intra-arterial, intra-articular, intracavitary, intraductal, intraglandular, intravascular, intranasal, intraperitoneal, intraspinal, epidural, intracranial, intracardial, intrapericardial, peritumoral, or intratumoral.

53. A method of providing access to a treatment site, the method comprising:
implanting in a subject a guide of claim 1, said implanting providing for placement of the guide distal end at a treatment site;
wherein the guide defines a conduit for access to the treatment site.
